(19) United States
(12) Patent Application Publication
(10) Pub. No.: US 2014/0259223 A1
(43) Pub. Date: Sep. 11, 2014

(54) METHODS FOR PRODUCING CINNAMOLIDE AND/OR DRIMENDIOL

(71) Applicant: Keygene N.V., Wageningen (NL)

(72) Inventors: Hendrik Jan Bouwmeester, Wageningen (NL); Maurice Gerard Leon Henquet, Wageningsen (NL); Maarten Anthonie Jongsma, Wageningen (NL)

(21) Appl. No.: 14/352,608

(22) PCT Filed: Oct. 19, 2012
(86) PCT No.: PCT/NL2012/050729

§ 371 Date: Apr. 17, 2014

Related U.S. Application Data
(60) Provisional application No. 61/548,934, filed on Oct. 19, 2011.

(30) Foreign Application Priority Data
Oct. 20, 2011 (NL) NL 2007626

(51) Int. Cl. C12N 9/04; C12N 15/82; C12P 7/02; C12P 17/04

(52) U.S. Cl. CPC C12N 9/0006; C12P 17/04; C12N 15/8286; C12P 7/02
USPC 800/279; 536/23.2; 435/190; 435/320.1; 435/325; 435/252.3; 435/254.11; 435/419; 800/298; 435/126; 800/278; 800/302; 435/155

(57) ABSTRACT

Described is a nucleic acid sequence isolated from Persicaria hydropiper and encoding a drimenol oxidase protein, expression vectors comprising such nucleic acid sequence, chimeric genes comprising such nucleic acid sequence, host cells or host organisms altered to harbour the drimenol oxidase nucleic acid sequence, and the drimenol oxidase protein itself. Methods for producing cinnamolide and/or drimendiol and/or enhanced levels of cinnamolide and/or drimendiol, in a cell or organism harbouring such nucleic acid sequence are provided. Transgenic organisms comprising the nucleic acid sequence or a chimeric gene of the invention are also provided. The present invention especially relates to transgenic plants with enhanced resistance to insects and enhanced insect antifeedant properties.

### FIELD OF THE INVENTION

The present invention is in the field of agrochemistry and agriculture. The invention particularly relates to a nucleic acid sequence isolated from Persicaria hydropiper and encoding a drimenol oxidase protein, expression vectors comprising such nucleic acid sequence, chimeric genes comprising such nucleic acid sequence, host cells or host organisms altered to harbour the drimenol oxidase nucleic acid sequence, and the drimenol oxidase protein itself. The invention further provides methods for producing cinnamolide and/or drimendiol and/or enhanced levels of cinnamolide and/or drimendiol, in a cell or organism harbouring such nucleic acid sequence, or by contacting the substrate drimenol with the drimenol oxidase polypeptide isolated from such cells or organisms. Transgenic organisms comprising the nucleic acid sequence or a chimeric gene of the invention are also provided. The present invention especially relates to transgenic plants with enhanced resistance to insects and enhanced insect antifeedant properties.

### BACKGROUND OF THE INVENTION

Pest insect management of crop plants represents a major concern in agriculture. Major efforts are devoted to develop methods to protect crop plants against pest insects and particularly against sap sucking pest insects. Sap sucking insects are able to feed on plants and transmit diseases by piercing the host tissue with their mouthpart, which allows them to tap into the phloem of plants. Such actions cause severe damage to the plant tissue and may translate into considerable economical losses for crop plants every year.

Commercially relevant examples of sap sucking insects include, but are not limited to, whiteflies and aphids and the like, which are considered among the most destructive insect pests on cultivated plants in temperate regions.

One strategy to control sap-sucking pest insects is the use of pesticides. However, the use of pesticides is expensive, and represents a serious hazard for the environment. Additionally, many pest insects rapidly gain resistance against synthetic pesticides, thus forcing the use of higher doses, which are often phytotoxic for the host plants and/or other organisms. Additionally, the mode of action of conventional pesticides is often non-specific. Thus, not only the pest insects of interest are eradicated but also beneficial insects in the environment.

Another strategy consists of the use of the so-called “semiochemicals”. Semiochemicals (or behaviour-modifying chemicals) modify insect behaviour in a stimulating or inhibiting manner depending on the situation. Such semiochemicals have greater specificity of action than pesticides, display low toxicity to vertebrates and invertebrates, display low phytotoxicity, low likelihood of accumulating and polluting the environment, biodegradability, and low likelihood of causing the insects to develop resistance through repeated exposures. Insect antifeedants inhibit normal feeding behaviour of insects and represent one category of semiochemicals. Many of the most potent insect antifeedants belong to the groups of monoterpenes, sesquiterpenes, and diterpenes. For instance, polygodial, warburganal, ugandensidial and muzigadial are prominent examples of plant sesquiterpenoid compounds endowed with insect antifeedant properties.

Overall these natural compounds further belong to a large and diverse class of naturally occurring organic chemicals, referred to as terpenoids. Plant terpenoids are synthesized from the common precursor isopentenyl pyrophosphate (IPP) and its isomer dimethylallyl diphosphate (DMAPP) through two distinct biosynthesis pathways: the mevalonate pathway found in the plant cytosol and in eukaryotes, and the 25 deoxyxylulose-5-phosphate (DXP) pathway found in the plant plastids and in prokaryotes. Generally, sesquiterpenes are synthesized from the relevant precursors through the mevalonate pathway in the cytosol, and monoterpenes and diterpenes are produced through the DXP pathway in plastids. Exchange of precursors between plastids and cytosol has also been observed. In both pathways, the IPP is further isomerized to DMAPP by the IPP isomerase with subsequent formation of the higher molecular weight acyclic polyprenyl pyrophosphate precursors by prenyl transferases to form the acyclic pyrophosphate terpene precursors. For example, these reactions produce ten-, fifteen-, and twenty-carbon precursors geranyl-pyrophosphate (GPP), farnesyl-pyrophosphate (FPP), geranylgeranyl-pyrophosphate (GGPP), respectively.

Terpene synthases are enzymes catalysing the cyclisation of acyclic precursors in the multi-step reactions producing the carbon skeleton of terpene, monoterpene or sesquiterpene compounds. For example, the initial step of the catalysed cyclisation may be the ionization of the diphosphate group to form an allylic cation. The substrate then undergoes isomerizations and rearrangements, which can be controlled by the active site of an enzyme. The product, for example, may be an acyclic, mono-, di or tricyclic terpene.

It is known in the art that GPP and neryl diphospate (NPP), the cis-isomer of GPP, are the substrates for monoterpene biosynthesis, and that FPP and GGPP are the respective substrates for sesquiterpene synthases and diterpene synthases. Some terpene synthases produce a single product, but many produce multiple products from the same precursor, or can produce multiple compounds depending on the precursor supplied (Van Schie et al., 2007 Plant Mol. Biol. Vol 64, pp. 251-263).

Several terpene synthases have been identified (WO2010/064897 and WO2009/044336). Previously, a partially purified protein from Polygonum (also known as Persicaria) hydropiper was identified as a drimenol cyclase (Banthorpe et al. 1992 Phytochemistry Vol 31: 3391). However, this reference did not provide the amino acid sequence of the protein, nucleotide sequence of a gene encoding it, or any methods for producing drimenol or derivatives thereof such as cinnamolide, polygodial and/or drimendiol.

The cytochrome P450 superfamily is a large and diverse group of enzymes that catalyze the oxidation of organic substances. Plant cytochromes P450 enzymes are involved in a wide range of biosynthetic reactions, leading to various fatty acid conjugates, plant hormones, defensive compounds, or medically important drugs. Terpenoids, which represent the largest class of characterized natural plant compounds, are often substrates for plant cytochrome P450 enzymes.

There is a need in the art to provide novel insect antifeedants. Such antifeedants may be isolated from natural sources. However, this approach is of little utility, due to the low content of antifeedant compounds, e.g., cinnamolide, and the difficulty of its isolation and purification. Additionally, there is a need in the art to identify novel ways of producing antifeedant compounds in amounts that cannot be obtained using isolation from natural resources, and to provide novel enzymes involved in the biosynthesis of antifeedants, particularly in the biosynthesis of drimenol oxidation products such as cinnamolide and/or drimendiol.

### SUMMARY OF THE INVENTION

The present invention provides a drimenol oxidase cDNA cloned from P. hydropiper and a drimenol oxidase protein that can be used for in vitro or in vivo production of drimenol oxidation products such as cinnamolide and/or drimendiol. Further, the present invention provides genetically modified host cells or genetically modified organisms capable of producing drimenol oxidase and/or enhanced levels of drimenol oxidase. The present invention also provide methods for the creation of said genetically modified host cells or host organisms and use thereof in the production of cinnamolide and/or drimendiol under in vivo and in vitro conditions.

A first aspect of the invention provides an isolated polypeptide having drimenol oxidase activity and comprising the amino acid sequence of SEQ ID NO:1, or an amino acid sequence that is at least 70%, 75%, 80%, 85%, 90%, 92%, 95%, 96%, 97%, 98% or 99% identical to the amino acid sequence of SEQ ID NO:1.

In a second aspect, an isolated nucleic acid sequence encoding such polypeptide, or variants, or fragments of such polypeptide is also provided. Such nucleic acid sequence includes the nucleotide sequence of SEQ ID NO:2 or a nucleotide sequence having at least 70%, 75%, 80%, 85%, 90%, 92%, 95%, 96%, 97%, 98% or 99% sequence identity with the nucleotide sequence of SEQ ID NO:2. In an embodiment, the isolated nucleic acid sequence is derived from P. hydropiper, and may e.g. be a cDNA sequence derived from P. hydropiper.

In another aspect of the invention, an expression vector is provided that includes a nucleic acid sequence encoding a polypeptide having drimenol oxidase activity and comprising the amino acid sequence of SEQ ID NO:1, or an amino acid sequence that is at least 70%, 75%, 80%, 85%, 90%, 92%, 95%, 96%, 97%, 98% or 99% identical to the amino acid sequence of SEQ ID NO:1.

In a further aspect of the invention a chimeric gene is provided comprising a nucleic acid encoding a polypeptide having drimenol oxidase activity and comprising the amino acid sequence of SEQ ID NO:1, or an amino acid sequence that is at least 70%, 75%, 80%, 85%, 90%, 92%, 95%, 96%, 97%, 98% or 99% identical to the amino acid sequence of SEQ ID NO:1.

In an embodiment of the invention, an expression vector is provided that includes the nucleotide sequence of SEQ ID NO:2, or a nucleotide sequence that is at least 70%, 75%, 80%, 85%, 90%, 92%, 95%, 96%, 97%, 98% or 99% identical to the nucleotide sequence of SEQ ID NO:2, or a chimeric gene comprising the nucleotide sequence of SEQ ID NO:2, or that includes a nucleotide sequence that is at least 70%, 75%, 80%, 85%, 90%, 92%, 95%, 96%, 97%, 98% or 99% identical to the nucleotide sequence of SEQ ID NO:2.

The expression vector and/or chimeric gene may comprise the nucleic acid sequence of the invention operably linked to at least one regulatory sequence which controls transcription, translation initiation or termination. E.g., the expression vector and/or the chimeric gene may comprise the nucleic acid sequence of the invention operably linked to at least one promoter. Said promoter may be constitutive or may be inducible, such as insect-inducible. Alternatively, said promoter may be tissue-specific.

In certain embodiments, the nucleic acid sequence of the present invention is further operably linked to a targeting sequence. For example, the targeting sequence may encode a transit peptide that targets the polypeptide product of the nucleic acid sequence of the present invention to a plastid of the plant cell. The plastid may be a chloroplast. Alternatively, the targeting sequence may encode a transit peptide that targets the polypeptide product of the nucleic acid sequence of the present invention to a mitochondrion of the plant cell.

In another aspect, a host cell is provided that comprises the isolated nucleic acid sequence of the present invention, or the chimeric gene as described above, and/or expression vector as described above, preferably wherein the cell is a prokaryotic or eukaryotic cell, such as a mammalian cell, a bacterial cell, a fungal cell, or a plant cell.

In another aspect, the host cell as described above is further capable of producing drimenol and/or drimenol synthase.

For example, the host cell as described above may further comprise a nucleotide sequence encoding a (drimenol synthase) polypeptide comprising the amino acid sequence of SEQ ID NO:3 or SEQ ID NO:4, or a variant thereof having at least 70%, such as at least 75%, 80%, 85%, 90%, 92%, 95%, 96%, 97%, 98% or 99%, sequence identity to the amino acid sequence of SEQ ID NO:3 or SEQ ID NO:4, or a chimeric gene or vector comprising a nucleotide sequence encoding a polypeptide comprising the amino acid sequence of SEQ ID NO: 3 or SEQ ID NO:4, or a variant of thereof having at least 70%, such as at least 75%, 80%, 85%, 90%, 92%, 95%, 96%, 97%, 98% or 99%, sequence identity with the amino acid sequence of SEQ ID NO:3 or SEQ ID NO:4, said polypeptide having drimenol synthase activity.

Alternatively, the host cell as described above may further comprise the nucleotide sequence of SEQ: ID NO:5, or SEQ ID NO:6, or a variant thereof having at least 70%, such as at least 75%, 80%, 85%, 90%, 92%, 95%, 96%, 97%, 98% or 99%, sequence identity to the nucleotide sequence of SEQ ID NO:5 or SEQ ID NO:6, or a chimeric gene comprising the nucleotide sequence of SEQ ID NO: 5 or SEQ ID NO:6, or a variant of thereof having at least 70%, such as at least 75%, 80%, 85%, 90%, 92%, 95%, 96%, 97%, 98% or 99%, sequence identity with the nucleotide sequence of SEQ ID NO:5 or SEQ ID NO:6.

In another aspect, a transgenic organism is provided comprising the isolated nucleic acid sequence of the present invention, the chimeric gene as described above, and/or expression vector as described above, preferably wherein the organism is a plant, more preferably a crop plant.

In a further aspect, the transgenic organism as described above is capable of producing drimenol and/or drimenol synthase.

In an embodiment, the transgenic organism as described above further comprises the nucleotide sequence of the present invention, or a chimeric gene as defined above.

In an embodiment, the transgenic organism as described above further includes any of the isolated nucleic acid sequences taught herein. For example, the transgenic organism may comprise a nucleotide sequence encoding a drimenol synthase polypeptide and may further comprise a nucleic acid sequence encoding a drimenol oxidase polypeptide. In general, the organism may be a plant, a micro-organism or a fungus. In a suitable embodiment of the invention, the organism is capable of producing drimenol and/or drimenol synthase.

In another aspect, the isolated polypeptide of the present invention, the isolated nucleic acid of the present invention, the chimeric gene as described above, the expression vector as described above, the host cell as described above, or the transgenic organism as described above, are used for producing cinnamolide and/or drimendiol.

In a further aspect, cinnamolide and/or drimendiol have insecticidal properties and/or (insect) antifeedant properties.

Another aspect of the invention is a method for producing cinnamolide and/or drimendiol, such method including the steps of: (a) contacting drimenol with a polypeptide having drimenol oxidase activity and comprising the amino acid sequence of SEQ ID NO:1, or an amino acid sequence that is at least 70%, 75%, 80%, 85%, 90%, 92%, 95%, 96%, 97%, 98% or 99% identical to the amino acid sequence of SEQ ID NO:1 to produce cinnamolide and/or drimendiol; and (b) isolating said cinnamolide and/or drimendiol.

Said method may further comprise prior to step (a) the step of transforming or transfecting a host cell or host organism capable of producing drimenol and/or drimenol synthase with a nucleic acid sequence encoding a polypeptide having drimenol oxidase activity and comprising the amino acid sequence of SEQ ID NO:1, or variants thereof, as described herein, or an expression vector and/or chimeric gene comprising such nucleic acid sequence.

In another embodiment, said method may further comprise prior to step (a) the step of transforming or transfecting a host cell or host organism capable of producing drimenol and/or drimenol synthase with the nucleotide sequence of SEQ ID NO:2, or variants as described herein, or an expression vector and/or chimeric gene comprising such nucleic acid sequence.

In one embodiment of the method of the invention, an additional step is comprised prior to step (a), said additional step being transforming or transfecting the host cell or host organism with a nucleic acid sequence encoding a polypeptide having drimenol synthase activity and comprising the amino acid sequence of SEQ ID NO:3 or SEQ ID NO:4, or a variant thereof having at least 70%, such as at least 75%, 80%, 85%, 90%, 92%, 95%, 96%, 97%, 98% or 99%, sequence identity with the amino acid sequence of SEQ ID NO:3 or SEQ ID NO:4, or an expression vector and/or chimeric gene comprising such nucleic acid sequence. In certain embodiments, such nucleic acid sequences encoding a polypeptide having drimenol synthase activity are derived from V. Officinalis and/or P. hydropiper.

In one embodiment, the method comprises an additional step prior to step (a), said additional step being transforming or transfecting the host cell or host organism with the nucleotide sequence of SEQ ID NO:5 or SEQ ID NO:6, or a variant thereof having at least 70%, such as at least 75%, 80%, 85%, 90%, 92%, 95%, 96%, 97%, 98% or 99%, sequence identity with the nucleotide sequence of SEQ ID NO:5 or SEQ ID NO:6, or an expression vector and/or a chimeric gene comprising such nucleotide sequence. In certain embodiments, such isolated nucleotide sequence is derived from V. Officinalis and/or P. hydropiper.

Step (a) may be carried out by culturing the host cell or organism under conditions permitting production of cinnamolide and/or drimendiol. The host cell may be selected from the group consisting of: prokaryotic and eukaryotic cells, such as mammalian cells, plant cells (including protoplasts), bacterial cells and fungal cells. In a preferred embodiment of the invention, the host cell is capable of producing drimenol and/or drimenol synthase.

Yet another aspect of the invention provides a method for producing a polypeptide having drimenol oxidase activity including the steps of: a) transforming or transfecting a host cell or a non-human organism with a nucleic acid sequence, chimeric gene, or the expression vector of the invention; and b) culturing the cell or the organism under conditions permitting production of cinnamolide and/or drimendiol. In a preferred embodiment of the method, the host cell or non-human organism is capable of producing drimenol and/or drimenol synthase.

In an embodiment, the host cell or non-human organism further comprises a nucleotide sequence encoding a polypeptide comprising the amino acid sequence of SEQ ID NO:3 or SEQ ID NO:4, or a variant thereof having at least 70%, such as at least 75%, 80%, 85%, 90%, 92%, 95%, 96%, 97%, 98% or 99%, sequence identity to the amino acid sequence of SEQ ID NO:3 or SEQ ID NO:4, or a chimeric gene comprising a nucleotide sequence encoding a polypeptide comprising the amino acid sequence of SEQ ID NO: 3 or SEQ ID NO:4, or a variant of thereof having at least 70%, such as at least 75%, 80%, 85%, 90%, 92%, 95%, 96%, 97%, 98% or 99%, sequence identity with the amino acid sequence of SEQ ID NO:3 or SEQ ID NO:4, said polypeptide having drimenol synthase activity.

In one embodiment, the host cell or the non-human cell further comprises the nucleotide sequence of SEQ: ID NO:5 or SEQ ID NO:6, or a variant thereof having at least 70%, such as at least 75%, 80%, 85%, 90%, 92%, 95%, 96%, 97%, 98% or 99%, sequence identity to the nucleotide sequence of SEQ ID NO:5 or SEQ ID No:6, or a chimeric gene comprising the nucleotide sequence of SEQ ID NO: 5 or SEQ ID NO:6, or a variant of thereof having at least 70%, such as at least 75%, 80%, 85%, 90%, 92%, 95%, 96%, 97%, 98% or 99%, sequence identity with the nucleotide sequence of SEQ ID NO:5 or SEQ ID NO:6.

In a further aspect, a method is provided for producing a transgenic plant capable of producing cinnamolide and/or drimendiol or capable of producing enhanced levels of cinnamolide and/or drimendiol compared to a non-transgenic plant of the same genetic background, the method comprising the steps of: a) transforming or transfecting a plant or a plant cell with a nucleic acid sequence, an expression vector and/or a chimeric gene of the present invention, and b) regenerating a plant. In an embodiment of the invention, said nucleic acid sequence is operably linked to at least one promoter, such as a constitutive promoter, an inducible promoter, or a tissue-specific promoter.

In an embodiment, the plant or plant cell is capable of producing drimenol and/or drimenol synthase.

In an embodiment, the plant or plant cell is further transformed or transfected with a nucleic acid sequence encoding a drimenol synthase polypeptide as defined herein. In certain embodiments, the isolated nucleic acid sequences encoding the drimenol synthase polypeptides taught herein are derived from V. officinalis and/or P. hydropiper.

In an embodiment, the method further includes the steps of screening the transgenic plant, or a plant derived therefrom by selfing or crossing, for production of cinnamolide and/or drimendiol, and identifying the plant producing cinnamolide and/or drimendiol. Such transgenic plant may be a crop plant.

In an embodiment, the transgenic plant may display insect resistance. The transgenic plant may have an enhanced insect resistance compared to a non-transgenic plant having the same genetic background but lacking the nucleic acid sequences taught herein.

In another aspect, the present invention related to use of cinnamolide and/or drimendiol as an antifeedant for insects, particularly sap-sucking insects.

### DETAILED DESCRIPTION OF THE INVENTION

### General Definitions

The terms “polypeptide” and “proteins” are used interchangeably and denote an amino acid sequence of consecutively polymerized amino acid residues, for instance, at least 15 residues, at least 30 residues, or at least 50 residues. In some embodiments of the invention, a polypeptide comprises an amino acid sequence that is an enzyme, or a fragment, or a variant thereof.

The term “isolated polypeptide” refers to an amino acid sequence that is removed from its natural environment by any method or combination of methods known in the art and includes recombinant, biochemical and synthetic methods.

The terms “drimenol synthase” “drimenol synthase protein”, and “drimenol synthase polypeptide” refer to an enzyme that is capable of converting farnesyl diphosphate (FPP) to drimenol, i.e. has drimenol synthase activity.

The terms “drimenol oxidase”, “drimenol oxidase protein”, and “drimenol oxidase polypeptide” refer to an enzyme that is capable of oxidising drimenol, e.g., to lead to production of cinnamolide and/or drimendiol, i.e., has drimenol oxidase activity.

The terms “biological function,” “function,” “biological activity” or “activity” when used in the context of the drimenol oxidase of the present invention, refer to the ability of the drimenol oxidase of the present invention to oxidise drimenol to form cinnamolide and/or drimendiol. The terms “biological function,” “function,” “biological activity” or “activity” when used in the context of the drimenol synthase as taught herein, refer to the ability of the drimenol synthase taught herein to catalyse the formation of drimenol from an FPP precursor.

The terms “nucleic acid sequence,” “nucleic acid,” “nucleotide sequence”, and “polynucleotides” are used interchangeably meaning a sequence of nucleotides. A nucleic acid sequence may be a single-stranded or double-stranded deoxyribonucleotide, or ribonucleotide of any length, and may include coding and non-coding sequences of a gene, exons, introns, sense and anti-sense complimentary sequences, genomic DNA, cDNA, miRNA, siRNA, mRNA, rRNA, tRNA, recombinant nucleic acid sequences, isolated and purified naturally occurring DNA and/or RNA sequences, synthetic DNA and RNA sequences, fragments, primers and nucleic acid probes. The skilled artisan is aware that the nucleic acid sequences of RNA are identical to the DNA sequences with the difference in thymine (T) being replaced by uracil (U).

An “isolated nucleic acid” or “isolated nucleic acid sequence” is defined as a nucleic acid or nucleic acid sequence that is in an environment different from that in which the nucleic acid or nucleic acid sequence naturally occurs, i.e. substantially separated from other cellular components, like ribosomes, polymerases and many other genome sequences which naturally accompany such nucleic acid in a cell in which it naturally occurs. The term “naturally-occurring” as used herein as applied to a nucleic acid refers to a nucleic acid that is found in a cell in nature. For example, a nucleic acid sequence that is present in an organism, for instance in the cells of an organism, that can be isolated from a source in nature and which has not been intentionally modified by a human in the laboratory is naturally occurring.

“Recombinant nucleic acid sequences” are nucleic acid sequences that result from the use of laboratory methods (molecular cloning) to bring together genetic material from more than one source, creating a nucleic acid sequence that does not occur naturally and would not otherwise be found in biological organisms.

“Recombinant DNA technology” refers to molecular biology procedures to join together nucleic acid sequences as described, for instance, in Laboratory Manuals edited by Weigel and Glazebrook, 2002 Cold Spring Harbor Lab Press; and Sambrook et al., 1989 Cold Spring Harbor, N.Y.: Cold Spring Harbor Laboratory Press.

The term “gene” means a DNA sequence comprising a region, which is transcribed into a RNA molecule, e.g., an mRNA in a cell, operably linked to suitable regulatory regions, e.g., a promoter. A gene may thus comprise several operably linked sequences, such as a promoter, a 5′ leader sequence comprising, e.g., sequences involved in translation initiation, a coding region of cDNA or genomic DNA, introns, and a 3′non-translated sequence comprising, e.g., transcription termination sites.

A “chimeric gene” or “recombinant gene” refers to any gene, which is not normally found in nature in a species, in particular, a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense, i.e., reverse complement of the sense strand, or inverted repeat sequence (sense and antisense, whereby the RNA transcript forms double stranded RNA upon transcription).

A “3′ UTR” or “3′ non-translated sequence” (also referred to as “3′ untranslated region,” or “3′end”) refers to the nucleic acid sequence found downstream of the coding sequence of a gene, which comprises for example a transcription termination site and (in most, but not all eukaryotic mRNAs) a polyadenylation signal such as AAUAAA or variants thereof. After termination of transcription, the mRNA transcript may be cleaved downstream of the polyadenylation signal and a poly(A) tail may be added, which is involved in the transport of the mRNA to the site of translation, e.g., cytoplasm.

“Homology” refers to a sequence similarity, or identity between a polypeptide or a fragment thereof and a reference sequence. A homology of polypeptide sequences are determined based on the number of amino acid sequences in the positions shared by the polypeptides. Homologous sequences may encompass amino acid sequences of the polypeptide of the present invention modified by chemical or enzymatic means known in the art. See Ausubel et al. (eds) 2000 Current Protocols Mol Biol, Willey & Sons, New York.

“Expression of a gene” involves transcription of the gene and translation of the mRNA into a protein. “Overexpression” refers to the production of the gene product as measured by levels of mRNA, polypeptide and/or enzyme activity in transgenic cells or organisms that exceeds levels of production in non-transformed cells or organisms of a similar genetic background.

“Expression vector” as used herein means a nucleic acid molecule engineered using molecular biology methods and recombinant DNA technology for delivery of foreign or exogenous DNA into a host cell. The expression vector typically includes sequences required for proper transcription of the nucleotide sequence. The coding region usually codes for a protein of interest but may also code for an RNA, e.g., an antisense RNA, siRNA and the like.

“Regulatory sequence” refers to a nucleic acid sequence that determines expression level of the nucleic acid sequences of the invention and that is capable of regulating the rate of transcription of the nucleic acid sequence that is operably linked to the regulatory sequence. Regulatory sequences comprise enhancers, transcription factors, promoter elements and the like.

“Promoter” refers to a nucleic acid sequence that controls the expression of a coding sequence by providing a binding site for RNA polymerase and other factors required for proper transcription, including, without limitation, transcription factor binding sites, repressor and activator protein binding sites. The term “promoter” also includes “promoter regulatory sequences.” Promoter regulatory sequences may include upstream and downstream elements that may influences transcription, RNA processing or stability of the associated coding nucleic acid sequence. Promoters include naturally-derived and synthetic sequences. The coding nucleic acid sequence is usually located downstream of the promoter with respect to the direction of the transcription starting at the transcription initiation site.

The term “constitutive promoter” refers to a promoter active in all or most tissues of a plant at all or most developmental stages.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous.

The term “target peptide” refers to a short (e.g., 3-60 amino acids long) peptide chain that directs the transport of a protein to a specific region in the cell, including the nucleus, mitochondria, endoplasmic reticulum (ER), chloroplast, apoplast, peroxisome and plasma membrane. A nucleic acid sequence encoding such target peptide is preferably operably linked to a coding sequence of a protein of interest, e.g., it may precede the coding sequence, e.g., be located in between a promoter sequence and the coding sequence in such a way that it is transcribed into a single mRNA construct with the coding sequence, the target peptide being located at the N-terminus of the protein coded for by the coding sequence. Some target peptides are cleaved from the protein by signal peptidases after the proteins are transported. The target peptide may be a peptide that is natively associated with the protein of interest, may replace a native target peptide, or may be entirely synthetic. The target peptide may be a (secretion) signal peptide, which directs the protein of interest it is associated with to the extracellular space.

The “% sequence identity” refers to a statistical measure of the degree of relatedness of two protein sequences, or of two nucleic acid sequences. The percentage of sequence identity between two sequences is determined using computer programs that are based on standard alignment algorithms. Sequences are substantially identical when they share at least a certain minimal percentage of sequence identity as identified by standard computer programs. Computer programs that are preferred within the scope of the present invention include without limitation the CGC program package (Devereux et al., 1984 Nucleic Acid Research 12:387), BestFit, BLASTP, BLASTN, and FASTA (Altshul et al., 1990 J. Mol. Biol. 215: 403), the algorithm of Meyers et al., 1988 Comput Appl Biosci 4:11, or the algorithm of Needleman et al., 1970 J. Mol. Biol. 48:443). Preferably, the sequence identity refers to the sequence identity over the entire length of the sequence.

The “primer” refers to a short nucleic acid sequence that is capable of hybridizing to a template nucleic acid sequence and is generally used for polymerization of a nucleic acid sequence complementary to the template.

As used herein, the terms “host cell”, “transformed cell” or “transfected cell” refer to a cell (or organism) altered to harbour at least one nucleic acid molecule as taught herein, for instance, a chimeric gene encoding a desired protein or a nucleic acid sequence which upon transcription yields a drimenol oxidase protein capable of producing cinnamolide and/or drimendiol. Also, the term “host cell” or “transformed cell” or “transfected cell” may refer to a cell (or organism) altered to harbour, for example, at least one nucleic acid molecule, for instance, a chimeric gene encoding a desired protein or a nucleic acid sequence which upon transcription yields a drimenol synthase protein capable of producing drimenol. The host cell may be selected from the group consisting of eukaryotic and prokaryotic cells, and is preferably a bacterial cell, a fungal cell or a plant cell. In an embodiment, the host cell is capable of producing drimenol and/or drimenol synthase. The host cell may comprise a chimeric gene as taught herein, which may have been integrated into the nuclear or organelle genomes of the host cell. Alternatively, the host cell may comprise the chimeric gene extra-chromosomally.

The term “selectable marker” refers to any gene which upon expression may be used to select a cell or cells that encompasses the selectable marker. Examples of selectable markers are described below. The skilled artisan will know that different antibiotic or herbicide selectable markers are applicable to different target species. Selectable markers that are routinely used in plant transformation include the npt II gene conferring resistance to kanamycin, paromymycin, geneticin, and related antibiotics (Veira and Messing, 1982 Gene 19: 259; Bevan et al., 1983 Nature 304: 184) the bacterial aadA gene encoding aminoglycoside 3′-adenyltransferase conferring resistance to antibiotics streptomycin or spectinomycin (Goldschmidt-Clermont, 1991 Nucl. Acid. Res. 19:4083), the hph gene conferring resistance to hygromycin (Blochlinger and Diggelmann, 1984 Mol Cell Bio I4: 2929). Other markers that can be used include a mutant EPSP gene conferring resistance to glyphosate (Hinchee et al. 1988 Biotechnology 6: 915), a mutant acetolactate synthase (ALS) gene conferring resistance to imidazoline or sulphonylurea herbicides (Lee at all., EMBO Journal 7: 1241), a phospinothricin acetyltransferase gene which confers resistance to herbicide phosphinothricin (White at al., 1990 Nucl Acid Res 18: 1062; Spencer et al., 1990 Theor. Appl. Genet. 79: 625). Selection markers resulting in positive selection such as phosphomannose isomerase gene are also used (see WO 93/05163).

The term “insect antifeedant” refers to a chemical compound which inhibits feeding but does not directly kill the insect. Rather, the insect often remains near the antifeedant source and is likely to die of starvation over time (Munakata, K. 1975, Appl. Chemistry vol 42, p. 57).

The term “drimenol” is used to denote any drimenol molecules having a formula C₁₅H₂₆O including (−)-drimenol (CAS: 468-68-8). As used herein, a “drimenol derivative” refers to any compound obtained from drimenol that has undergone one or more steps of hydroxylation, oxidation, acetylation, isomerization, dimethylation, and the like, and containing essential elements of the parent substance, and includes without limitation driman-8-ol, driman-8,11-diol, drim-8-en-7-one, forskolin, cinnamodial, (+)-albicanol, (−)-uvidin, (+)-isopolygonal, (−)-polygodial, (−)-ugandensidial, (−)-warburganal, ambergris, drimenal, drimenoic acid, isodrimenin, cinnamolide, confertolin, drimendiol, and polygodial acid.

The terms “cinnamolide” and “drimendiol” are used to denote any cinnamolide molecules having a formula C₁₅H₂₂O₂ including (−)-cinnamolide (CAS: 235-99-47-5) and drimendiol molecules having a formula (C₁₅H₂₆O₂) (−)-drimendiol (CAS: 34437-62-2), respectively. It is not known how cinnamolide and drimendiol are produced in nature but it is believed to be produced through a series of appropriate cyclizations, oxidations and rearrangements from FPP. The enzyme drimenol synthase converts FPP to drimenol. Subsequently, a drimenol oxidase is involved oxidising drimenol and converting drimenol to cinnamolide and/or drimendiol.

Similarly, the term “polygodial” refers to any type of polygodial molecule of a formula C₁₅H₂₂O₂. In nature, polygodial is made in at least two steps from FPP, first by the enzyme drimenol synthase, and subsequently by a cytochrome P450 enzyme to first introduce a hydroxylation and subsequently oxidation into two aldehydes on the drimenol backbone (Pickett 1985, Production of behavior-controlling chemicals by crop plants. Phil Transactions of the Royal Society of London Vol 310, pp. 235-239).

The term “organism” refers to any non-human multicellular or unicellular organisms such as, without limitation, a plant, or a microorganism. The microorganism may be selected from the group consisting of a bacterium, a fungus, or a yeast. Advantageously, the micro-organism is capable of producing drimenol and/or drimenol synthase.

The term “plant” is used interchangeably to include whole plants, plant tissues, plant cells, plant protoplasts, plant cell tissue cultures giving rise to regenerated plants, or parts of plants, or plant organs such as roots, stems, leaves, flowers, pollen, ovules, embryos, fruits and the like. Any plant can be used to carry out the methods of the invention. Preferably, the plant is selected from the family Solanaceae, Valerianaceae, Malvaceae, Asteraceae, Brassicaceae, Polygonaceae, Poaceae (formerly Gramineae) or Fabaceae. The term “plant cell” as used herein includes plant protoplast.

The term “crop species” refers to plants cultivated for purposes of obtaining food, feed or plant derived products including carbohydrates, oils and medicinal ingredients.

As used herein, a “genetic background” refers to the genotypic base of a plant breeding line or population of organisms.

The terms “plant insects” or “plant pests” or “pest insects” refer to insect species that infest and damage plants, such as crop and ornamental plants. An “infestation” refers to presence of a large number of pest insects in a field or greenhouse, on the surface of a host plant or on anything that might contact a host plant, or in the soil. Pest insects include sap-sucking pest insect such as psyllids, whiteflies, aphids, mealybugs, plant hoppers, and scale insects which share a common property, namely the utilization of plant sap as their food source. Pest insects also include thrips, cicada, mites, and leaf hoppers. The term “pest insects” also refers herein to insects of the order Diptera including but not limiting to blood sucking or biting insects attacking animals, especially mammals. Blood sucking ticks are also included. Such insects or arachnids may act as vectors of human and/or mammalian diseases such as malaria.

The term “whitefly” or “whiteflies” refers to species of the genus Bemisia, especially B. tabaci, species of the genus Trialeurodes, especially the greenhouse whitefly T. vaporariorum and the banded winged whitefly T. abutinolea. All biotypes of B. tabaci such as biotype Q and B, are also included as well as any developmental stage, such as eggs, larvae, pupae and adults.

As used herein, the term “aphids” refers to plant pest insects belonging to the family Aphididae, including but not limited to Aphis gossypii, A. fabae, A. glycines, A. nerii, A. nasturtii, Myzus persicae, M. cerasi, M. ornatus, Nasonovia especially N. ribisnigri, Macrosiphum, and Brevicoryne.

“Insect vectors” are insects that are capable of carrying and transmitting viruses to plants. In the context of mammalian disease vectors, insect vectors are insects which attack mammals and can potentially transmit diseases to mammals, such as mosquitoes, which are able to transmit the parasite Plasmodium to humans or heartworm to canines. Preferably, the modified plants of the invention develop enhanced resistance to one or more pest insects.

In this document and in its claims, the verb “to comprise” and its conjugations is used in its non-limiting sense to mean that items following the word are included, but items not specifically mentioned are not excluded. It also encompasses the more limiting verb “to consist of”. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

### Polypeptides of the Invention

It is an object of the present invention to provide a novel drimenol oxidase protein.

In an aspect, the present invention provides a drimenol oxidase polypeptide, drimenol oxidase homologous polypeptides, and variants thereof. The polypeptides of the invention herein are capable of oxidising drimenol to form cinnamolide and/or drimendiol. The present inventors have found that further intermediates in the biosynthesis pathway for cinnamolide and/or polygodial are also formed upon oxidisation of drimenol using the polypeptide of the present invention. Such further intermediates include, without limitation, the compounds as identified by their mass fragmentation spectrum in any one of Panels A-E of FIG. 1B as well as those in any one of Panels F-K of FIG. 4B. These compounds could not be identified with a NIST library search. As such, it is believed that also polygodial is formed by the oxidation of drimenol accomplished by the polypeptide of the present invention. Whereas polygodial itself could not be identified as a product formed by the action of the polypeptide of the present invention, it is well-known that polygodial is prone to form adducts and conjugates. It is believed that some of the mass spectra displayed in FIGS. 1B and 4B may represent such adducts and/or conjugates.

Thus, the invention provides an isolated or recombinant polypeptide having drimenol oxidase activity and comprising the amino acid sequence set forth in SEQ ID NO:1, or fragments, variants or derivatives thereof. Ordinarily, such variants will comprise at least 55% sequence identity to the amino acid sequence of SEQ ID NO:1, preferably over the entire length of the polypeptide as represented by SEQ ID NO:1. Preferably, the variants will comprise at least 55%, 60%, 65%, 70%, 75%, 80%, 85%, 90%, 92%, 95%, 96%, 97%, 98% or 99% sequence identity to the amino acid sequence of SEQ ID NO:1, preferably over its full length.

Fragments of a polypeptide comprising the amino acid sequence of SEQ ID NO:1 are subsequences of the polypeptide of the invention that retain drimenol oxidase activity and capacity to oxidise drimenol to form cinnamolide and/or drimendiol from drimenol. The term “fragment” may refer to a recombinant polypeptide and/or an aggregate polypeptide such as a dimer or multimer. Fragments of the drimenol oxidase protein of the present invention comprise fragments of 100, 150, 200, 300, 400, 500 contiguous amino acids or more of the polypeptide or variants or derivatives thereof. Preferably fragments retain drimenol oxidase activity in non-human organisms and are capable of producing cinnamolide and/or drimendiol from drimenol in a host cell or non-human organism. In a preferred embodiment, the host cell or non-human organism is capable of producing drimenol and/or drimenol synthase.

Also included are sequences which share homology with the polypeptides having the amino acid sequence of SEQ ID NO:1. Homologous sequences are sequences that share substantial sequence identity or similarity to the amino acid sequence of SEQ ID NO:1, which retain drimenol oxidase activity when (over) expressed, or ectopically expressed in a plant. Polypeptide sequences that are at least 50%, such as at least 55%, 60%, 65%, 70%, 75%, 80%, 85%, 90%, 92%, 95%, 96%, 97%, 98% or 99%, identical to the polypeptide of the present invention are considered sufficiently identical. Such homologous sequences may be derived from any plants including monocots or dicots, and especially crops plants including but not limited to tomato, pepper, eggplant, lettuce, sunflower, oilseed rape, broccoli, cauliflower and cabbage crops, cucumber, melon, watermelon, pumpkin, squash, peanut, soybeans, cotton, beans, avocado, onion, endive, leek, roots such as arrowroot, carrot, beet, turnip, radish, yam, cassava, potatoes, sweet potatoes and okra. Homologous sequences may also be derived from crop species including maize, barley, pearl millet, wheat, rye, sorghum, rice, tobacco and forage grasses. Homologous sequences may further be derived from tree species and fleshy fruit species such as lemons, tangerines, oranges, grapes, peaches, plums, currant, cherries, melons, strawberry, and mango, or from ornamental plant species such as hibiscus, poinsettia, lily, iris, rose and petunia, and the like. Additionally, homologous sequences may be derived from plant species that are wild relatives of crop plant species. For example, homologous sequences may be derived from nightshade Atropa belladonna which is a wild relative of a cultivated tomato Solanum lycopersicum, or teosinte species related to maize.

Homologous sequences include orthologous or paralogous sequences. Methods of identifying orthologs or paralogs including phylogenetic methods, sequence similarity and hybridization methods, are known in the art and are described herein.

Paralogs result from gene duplication that gives rise to two or more genes with similar sequences and similar functions. Paralogs typically cluster together and are formed by duplications of genes within related plant species. Paralogs are found in groups of similar genes using pair-wise Blast analysis (Feng and Dollitle, 1987 J Mol Evol: 25:351) or during phylogenetic analysis of gene families using programs such as CLUSTAL (Thompson et al. 1994 Nucl Acid Res 22:4573; Higgins et al., 1996 Methods Enzymol 266:383). In paralogs, consensus sequences can be identified characteristic to sequences within related genes and having similar functions of the genes.

Orthologs, or orthologous sequences, are sequences similar to each other because they are found in species that descended from the common ancestor. For instance, plant species that have common ancestors are known to contain many enzymes that have similar sequences and functions. The skilled artisan can identify orthologous sequences and predict the functions of the orthologs, for example, by constructing a polygenic tree for a gene family of one species using CLUSTAL or BLAST programs. A method for identifying or confirming similar functions among homologous sequences is by comparing of the transcript profiles in plants overexpressing or lacking (in knockouts/knockdowns) related polypeptides. The skilled person will understand that genes having similar transcript profiles, with greater than 50% regulated transcripts in common, or with greater than 70% regulated transcripts in common, or greater than 90% regulated transcripts in common will have similar functions. Homologs, paralogs, orthologs and any other variants of the sequences herein are expected to function in a similar manner by making plants producing drimenol oxidase proteins.

An embodiment of the invention provides amino acid sequences of drimenol oxidase proteins including orthologs and paralogs as well as methods for identifying and isolating orthologs and paralogs of the drimenol oxidases in other organisms. Preferably, so identified orthologs and paralogs of the drimenol oxidase retain drimenol oxidase activity and are capable of producing cinnamolide and/or drimendiol starting from drimenol.

In yet another embodiment, a “variant” or “derivative” of the polypeptide as set forth in SEQ ID NO:1 is provided, such variant or derivative being a polypeptide with substantial similarity with the amino acid sequence of the polypeptide herein, e.g. at least 70%, 75%, 80%, 85%, 90%, 92%, 95%, 96%, 97%, 98% or 99% identical to the amino acid sequence of SEQ ID NO:1. The amino acid sequences of the polypeptide of the invention and variants thereof may differ by deletions, additions, and/or substitutions of amino acids while retaining functional equivalence to the polypeptide (i.e. drimenol oxidase activity, e.g., to form cinnamolide and/or drimendiol). For instance, amino acids of the polypeptide of the invention may be modified based on similarity in hydrophobicity, hydrophilicity, solubility, polarity of amino acid residues, as long as the variant polypeptide remains functionally equivalent to the polypeptide of the invention, i.e, is capable of oxidising drimenol as evidenced by formation of cinnamolide and/or drimendiol. Variants also include proteins having drimenol oxidase activity, which have been derived, by way of one or more amino acid substitutions, deletions or insertions, from the polypeptide having the amino acid sequence of SEQ ID NO:1. Preferably, such proteins comprise from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions.

A variant may, for example, differ from the polypeptide of the invention by attachment of modifying groups which are covalently or non-covalently linked to the polypeptide backbone. The variant also includes a polypeptide which differs from the polypeptide of the present invention by introduced N-linked or O-linked glycosylation sites, and/or an addition of cysteine residues. The skilled artisan will recognize how to modify an amino acid sequence and preserve biological activity.

The functionality or activity of any drimenol oxidase protein, variant or fragment of the present invention, may be determined using various methods. For example, transient or stable (over) expression in plant, bacterial or yeast cells can be used to test whether the protein has activity, i.e., oxidises drimenol to produce, e.g., cinnamolide and/or drimendiol. Drimenol oxidase activity may be assessed in a yeast expression system, such as the assay described in Example 2 (see FIGS. 4 and 5) herein on the production of cinnamolide and/or drimendiol, indicating functionality. Alternatively, functionality or activity of any drimenol oxidase protein of the invention may be determined using transient expression in a plant, such as Nicotiana benthamiana, e.g., using the assay as described in Examples 3 and 4. The skilled person will routinely be able to determine functionality or activity of the drimenol oxidase protein of the present invention. Variants of the drimenol oxidase of the present invention may have additional desirable biological functions including, e.g., altered substrate utilization, reaction kinetics, product distribution or other alterations.

### Methods Utilizing the Polypeptide of the Invention

An embodiment herein provides polypeptides of the invention to be used in a method to produce cinnamolide and/or drimendiol and/or intermediates of the cinnamolide and/or polygodial biosynthesis pathway from drimenol, e.g., those having a mass fragmentation spectrum as determined by GC-MS essentially as shown in any one of panels A-E of FIG. 1B or any one of panels F-L of FIG. 4B and/or drimenol oxidation products.

In one aspect, the present invention pertains to a method for producing cinnamolide and/or drimendiol, comprising the step of contacting drimenol with a polypeptide of the invention under in vitro or in vivo conditions. In an embodiment, the method comprises the steps of: a) contacting drimenol with a polypeptide of the present invention, preferably under conditions allowing oxidation of drimenol to form cinnamolide and/or drimendiol; and b) isolating said cinnamolide and/or drimendiol.

To carry out the in vitro method, the polypeptide comprising the amino acid sequence of SEQ ID NO:1, or variants, or fragments thereof as set forth above, e.g. those that comprise at least 55%, 60%, 65%, 70%, 75%, 80%, 85%, 90%, 92%, 95%, 96%, 97%, 98% or 99%, sequence identity, preferably over the entire length of the polypeptide of the invention, may be isolated from any organism expressing such polypeptide, for instance, after transformation or transfection with the polynucleotides of the invention. It is known in the art that a cell can be transformed or transfected with a nucleic acid encoding a desired protein to be secreted, for instance, to the culture medium, as to produce large quantities of the protein. The protein can be collected from the culture medium and further used, for example, to produce cinnamolide and/or drimendiol from drimenol as a starting compound.

In an alternative embodiment, the cell or cells may be engineered to accumulate or overaccumulate the polypeptide of the invention within the cell. The skilled person will recognize how to extract proteins or polypeptides from the cell, for instance, by using the following techniques: repeated freezing and thawing, sonication, homogenization by high pressure, filtration, or permeabilization by organic solvents, and the like. After the extraction, proteins can be re-suspended in a buffer solution at optimal pH and then drimenol may be added to the solution to produce cinnamolide and/or drimendiol. After incubation, the resultant cinnamolide and/or drimendiol may be removed from the solution by standard isolation and purification procedures, e.g., as set forth in the examples section.

It may be particularly advantageous to direct the localization of the drimenol oxidase proteins to a subcellular compartment, for example, to plastids, preferably chloroplasts, mitochondria, endoplasmic reticulum, or vacuoles. Targeting of proteins to the particular cell structure may provide most efficient functioning for the desired expressed proteins. Putative transit peptides can be detected using computer based analysis, using programs such as the program Signal Peptide search (SignalP V3.0; Von Heijne, Gunnar, 1986 and Nielsen et al. 1996). It is well known in the art that proteins can be directed to the chloroplast by including at their N-terminus a chloroplast transit peptide. Naturally occurring chloroplast targeted proteins, synthesized as larger precursor proteins containing an N-terminal chloroplast targeting peptide directing the precursor to the chloroplast import machinery, have been previously identified and are well-known in the art. Chloroplast targeting peptides have been found particularly useful for designing plants capable of overproducing terpenes which were observed to be toxic to the host if overexpressed in cytosol but not in plastids.

Sequences which are suitable for directing the targeting of the drimenol oxidase gene product to mitochondria, such as the Cox IV targeting signal (Kohler et al. 1997 Plant J 11: 61) are also within the scope of the invention herein. To allow secretion of the drimenol oxidase proteins to the outside of the transformed or transfected host cell or organism, an appropriate secretion signal peptide may be fused to the amino terminal end, e.g., N-terminal end, of the drimenol oxidase protein. Such secretion signal peptide includes, without limitations, that of the potato proteinase inhibitor II (Keil et al. 1986 Nucl Acids Res 14: 5641), the secretion signal of the alpha-amylase 3 gene of rice (Sutliff et al. 1991 Plant Mol Biol 16:579) and the secretion signal of tobacco PR1 protein (Cornelissen et al. 1986 EMBO J 5:37).

Particularly useful transit peptides in accordance with the invention include the chloroplast transit peptide (Van Den Broeck et al. 1985 Nature 313:358), or the optimized chloroplast transit peptide (U.S. Pat. No. 5,510,471 and U.S. Pat. No. 5,635,618) causing transport of the protein to the chloroplasts, a secretory signal peptide or a peptide targeting the protein to other plastids, mitochondria, the ER, or another organelle.

Besides targeting polypeptides of the invention to intracellular organelles, methods of transformation of the plastid genome, preferably chloroplast genome, or mitochondrial genome are also included in the invention. Transformation of organelles is known to the skilled artisan and provides means to control environmental transgene spread (Sidorov et a., 1999 Plant J 19: 209; Lutz et al. 2004 Plant J 37: 906).

### Polynucleotides of the Invention

The present invention also provides an isolated polynucleotide encoding the drimenol oxidase polypeptide or variants or fragments thereof provided herein.

In an embodiment of the invention, an isolated, recombinant or synthetic nucleic acid is provided comprising the nucleotide sequence of SEQ ID NO:2, or a variant thereof which is at least 70%, 75%, 80%, 85%, 90%, 92%, 95%, 96%, 97%, 98% or 99% identical to the nucleotide sequence of SEQ ID NO:2, and which encodes a polypeptide having drimenol oxidase activity of the present invention including variants or fragments. Variants of the nucleotide sequence of SEQ ID NO:2 also include nucleic acid sequences hybridizing to the complement of drimenol oxidase encoding nucleic acid sequences under stringent hybridization conditions and having drimenol oxidase activity as defined herein.

Embraced by the present invention are cDNA, genomic DNA and RNA sequences. Any nucleic acid sequence encoding the drimenol oxidase of the present invention or variants thereof is referred herein as a drimenol oxidase encoding sequence. According to a preferred embodiment, the nucleotide sequence of SEQ ID:2 is the coding sequence of the drimenol oxidase gene encoding the drimenol oxidase obtained from P. hydropiper as described in the Examples herein. In an embodiment, the isolated nucleic acid sequence of the invention is derived from P. hydropiper.

A fragment of a polynucleotide of SEQ ID NO:2, or variants thereof, refers to a stretch of contiguous nucleotides that is preferably at least 15 bp, at least 30 bp, at least 40 bp, at least 50 bp and/or at least 60 bp in length of the polynucleotide of the invention herein. Preferably the fragment of a polynucleotide comprises at least 25, more preferably at least 50, more preferably at least 75, more preferably at least 100, more preferably at least 150, more preferably at least 200, more preferably at least 300, more preferably at least 400, more preferably at least 500, more preferably at least 600, more preferably at least 700, more preferably at least 800, more preferably at least 900, more preferably at least 1000 contiguous nucleotides of the polynucleotide of the invention. Without being limited, the fragment of the polynucleotides herein may be used as a PCR primer, and/or as a probe, or for anti-sense gene silencing or RNAi.

It is clear to the person skilled in the art that genes, including the polynucleotides of the invention, can be cloned on basis of the available nucleotide sequence information, such as found in the attached sequence listing, by methods known in the art. These include e.g. the design of DNA primers representing the flanking sequences of such gene of which one is generated in sense orientations and which initiates synthesis of the sense strand and the other is created in reverse complementary fashion and generates the antisense strand. Thermo stable DNA polymerases such as those used in polymerase chain reaction are commonly used to carry out such experiments. Alternatively, DNA sequences representing genes can be chemically synthesized and subsequently introduced in DNA vector molecules that can be multiplied by e.g. compatible bacteria such as e.g. E. coli.

In a related embodiment of the invention, PCR primers and/or probes for detecting nucleic acid sequences encoding a drimenol oxidase of the present invention are provided. The skilled artisan will be aware of methods to synthesize degenerate or specific PCR primer pairs to amplify a nucleic acid sequence encoding the drimenol oxidase or fragments thereof, based on the nucleotide sequence of SEQ ID NO:2, or variants thereof, (see Dieffenbach and Dveksler, 1995 PCR Primer: A Laboratory Manual, Cold Spring Harbor Laboratory Press; McPherson et al., 2000 PCR Basics: From Background to Bench, 1^{st} ed., Springer Verlag, Germany). A detection kit for nucleic acid sequences encoding the drimenol oxidase, or variants thereof, may include primers and/or probes specific for nucleic acid sequences encoding the drimenol oxidase, or variants thereof as taught herein, and an associated protocol to use the primers and/or probes to detect nucleic acid sequences encoding the drimenol oxidase, or variants thereof as taught herein, in a sample. Such detection kits may be used to determine whether a plant has been modified, i.e., transformed or transfected with a nucleic acid sequence encoding the drimenol oxidase or variants thereof as taught herein.

Due to the degeneracy of the genetic code, more than one codon may encode the same amino acid sequence, multiple nucleic acid sequences can code for the same protein or polypeptide. Where appropriate, the nucleic acid sequences encoding the drimenol oxidase may be optimized for increased expression in the host cell. For example, nucleotides of the invention may be synthesized using codons preferred by a host for improved expression (See Campbell and Gowri 1990 Plant Physiol 92:1; Bennetzen and Hall 1982 J Biol Chem 257:3026). Methods are available in the art for constructing plant-preferred synthetic DNA sequences (see U.S. Pat. Nos. 5,380,831 and 5,436,391). Codon usage tables for plant species are publicly available (see Ikemura 1993 In Plant Molecular Biology Labfax, Croy ed., Bios Scientific Publishers Ltd, pp. 37-48; Codon Usage Database at the Kazusa DNA Research Institute, Japan).

A stringent hybridization is performed at a temperature 65° C. and most preferably at 55° C. in double strength (2×) citrate buffered saline (SSC) containing 0.1% SDS followed by rinsing of the support at the same temperature but with a buffer having reduced SSC concentration. Such reduced concentration buffers are typically one tenth strength SSC (0.1×SSC) containing 0.1% SDS, preferably 0.2×SSC containing 0.1% SSC and most preferably half strength SSC (0.5×SSC) containing 0.1% SDS. Functional equivalents of the drimenol oxidase proteins from other organisms can be found by hybridizing a nucleic acid sequence with the nucleic acid sequence of SEQ ID NO:2 with genomic DNA isolated from other organisms.

The skilled artisan will be aware of methods to identify homologous sequences in other organisms and methods (identified in the Definition section herein) to determine the percentage of sequence identity between homologous sequences. Such newly identified DNA molecules then can be sequenced and the sequence can be compared with the nucleic acid sequence of SEQ ID NO:2 and tested for functional equivalence. Within the scope of the present invention are DNA molecules having at least 70%, preferably at least 80%, more preferably at least 90% and most preferably at least 95% or more sequence identity to the nucleotide sequence of SEQ ID NO:2.

A related embodiment of the invention provides a nucleic acid sequence which is complementary to the nucleic acid sequence of SEQ ID NO:2, such as inhibitory RNAs, or a nucleic acid sequence which hybridizes under stringent conditions to at least part of the sequence according to the nucleotide sequence of SEQ ID NO:2 or SEQ ID NO:5 or SEQ ID NO:6.

### Chimeric Genes, Expression Vectors, Host Cells, and Transgenic Organisms

In one embodiment of the invention nucleic acid sequences encoding drimenol oxidase proteins (including variants or fragments), as described above, are used to make chimeric genes, and expression vectors comprising these for transfer of the nucleic acid sequence or chimeric gene of the present invention into a host cell and production of the drimenol oxidase protein(s) in host cells, such as cells, tissues, organs or organisms derived from transformed cell(s). In an embodiment, the drimenol oxidase of the present invention is employed for the production of cinnamolide and/or drimendiol. Vectors for the production of drimenol oxidase protein (or protein fragments or variants) in plant cells are herein referred to as “expression vectors”.

Suitable host cells for expression of polypeptides such as drimenol oxidase include prokaryotes, yeast, or higher eukaryotic cells. Appropriate cloning and expression vectors for use with bacterial, fungal, yeast, and mammalian cellular hosts are described, for example, in Pouwels et al., Cloning vectors: A Laboratory Manual, Elsevier, N.Y., (1985). Cell-free translation systems could also be employed to produce the proteins of the present invention using RNAs derived from nucleic acid sequences disclosed herein. In a suitable embodiment, said host cell produces or overproduces drimenol and/or drimenol synthase. The skilled person is capable of overproducing the substrate of the drimenol oxidase of the present invention to produce cinnamolide and/or drimendiol.

Suitable prokaryotic host cells include gram-negative and gram-positive organisms, for example, Escherichia coli or Bacilli. Another suitable prokaryotic host cell is Agrobacterium, in particular Agrobacterium tumefaciens.

Proteins of the present invention can also be expressed in yeast host cells, for example from the Saccharomyces genus (e.g., Saccharomyces cerevisiae). Other yeast genera, such as Pichia or Kluyveromyces, can also be employed.

Alternatively, proteins of the present invention may be expressed in higher eukaryotic host cells, including plant cells, fungal cells, insect cells, and mammalian, optionally non-human, cells.

One embodiment of the invention is a non-human organism modified to comprise a nucleic acid sequence of the present invention (transgenic organism). The non-human organism and/or host cell may be modified by any methods known in the art for gene transfer including, for example, the use of delivery devices such as lipids and viral vectors, naked DNA, electroporation, chemical methods and particle-mediated gene transfer. In an advantageous embodiment, the non-human organism is a plant.

Any plant may be a suitable host, such as monocotyledonous plants or dicotyledonous plants, but most preferably the host plant belongs to the family Solanaceae. For example, the plant may belong to the genus Solanum (including Lycopersicon), Nicotiana, Capsicum, Petunia and other genera. The following host species may suitably be used: Tobacco (Nicotiana species, e.g. N. benthamiana, N. plumbaginifolia, N. tabacum, etc.), vegetable species, such as tomato (L. esculentum, syn. Solanum lycopersicum) such as e.g. cherry tomato, var. cerasiforme or currant tomato, var. pimpinellifolium) or tree tomato (S. betaceum, syn. Cyphomandra betaceae), potato (Solanum tuberosum), eggplant (Solanum melongena), pepino (Solanum muricatum), cocona (Solanum sessiliflorum) and naranjilla (Solanum quitoense), peppers (Capsicum annuum, Capsicum frutescens, Capsicum baccatum), ornamental species (e.g. Petunia hybrida, Petunia axillaries, P. integrifolia), coffee (Coffea).

Alternatively, the plant may belong to any other family, such as to the Cucurbitaceae or Gramineae. Suitable host plants include for example maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), cucumber, artichoke, asparagus, broccoli, garlic, leek, lettuce, onion, radish, turnip, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa), or model organisms, such as Arabidopsis thaliana.

Preferred hosts are “crop plants” or “cultivated plants”, i.e. plant species which are cultivated and bred by humans. A crop plant may be cultivated for food or feed purposes (e.g. field crops), or for ornamental purposes (e.g. production of flowers for cutting, grasses for lawns, etc.). A crop plant as defined herein also includes plants from which non-food products are harvested, such as oil for fuel, plastic polymers, pharmaceutical products, cork, fibres (such as cotton) and the like.

The construction of chimeric genes and vectors for, preferably stable, introduction of nucleic acid sequences into the genome of host cells is generally known in the art. To generate a chimeric gene a nucleic acid sequence, e.g., one encoding a drimenol oxidase protein (or variant or fragment thereof), is operably linked to a promoter sequence, suitable for expression in the host cells, using standard molecular biology techniques. The promoter sequence may already be present in a vector so that the drimenol oxidase nucleic acid sequence is simply inserted into the vector downstream of the promoter sequence. The vector is then used to transform the host cells and the chimeric gene is inserted in the nuclear genome or into the plastid, mitochondrial or chloroplast genome and expressed there using a suitable promoter (e. g., Mc Bride et al., 1995 Bio/Technology 13, 362; U.S. Pat. No. 5,693,507). In one embodiment, a chimeric gene comprises a suitable promoter for expression in plant cells or microbial cells (e.g. bacteria), operably linked to a nucleic acid sequence encoding a drimenol oxidase protein according to the invention, optionally followed by a 3′nontranslated nucleic acid sequence. The bacteria may subsequently be used for plant transformation (Agrobacterium-mediated plant transformation).

The drimenol oxidase encoding nucleic acid sequence, such as a drimenol oxidase encoding chimeric gene, encoding an functional drimenol oxidase protein of the invention (including variants or fragments) can be stably inserted in a conventional manner into the nuclear genome of a single plant cell, and the thus transformed plant cell can be used in a conventional manner to produce a transformed plant that has an altered phenotype due to the presence of the drimenol oxidase protein in certain cells at a certain time. In this regard, a T-DNA vector, comprising a nucleic acid sequence encoding a drimenol oxidase protein, in Agrobacterium tumefaciens can be used to transform the plant cell, and thereafter, a transformed plant can be regenerated from the transformed plant cell using the procedures described, for example, in EP 0 116 718, EP 0 270 822, PCT publication WO84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Preferred T-DNA vectors each contain a promoter operably linked to a drimenol oxidase encoding nucleic acid sequence of the invention (e.g. SEQ ID NO: 2) between T-DNA border sequences, or at least located to the left of the right border sequence. Border sequences are described in Gielen et al. (1984, EMBO J 3, 835-845). Of course, other types of vectors can be used to transform the plant cell, using procedures such as direct gene transfer (as described, for example in EP 0 223 247), pollen mediated transformation (as described, for example in EP 0 270 356 and WO85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods. For tomato or tobacco transformation see also An G. et al., 1986, Plant Physiol. 81: 301-305; Horsch R. B. et al., 1988, In: Plant Molecular Biology Manual A5, Dordrecht, Netherlands, Kluwer Academic Publishers. pp 1-9; Koornneef M. et al., 1986, In: Nevins D. J. and R. A. Jones, eds. Tomato Biotechnology, New York, N.Y., USA, Alan R. Liss, Inc. pp 169-178). For potato transformation see e.g. Sherman and Bevan (1988, Plant Cell Rep. 7: 13-16).

Likewise, selection and regeneration of transformed plants from transformed cells is well known in the art. Obviously, for different species and even for different varieties or cultivars of a single species, protocols are specifically adapted for regenerating transformants at high frequency.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov V A et al. 1999, Plant J. 19: 209-216 or Lutz K A et al. 2004, Plant J. 37(6):906-13.

The resulting transformed plant can be used in a conventional plant breeding scheme to produce more transformed plants containing the transgene. Single copy transformants can be selected, using e.g. Southern Blot analysis or PCR based methods or the Invader® Technology assay (Third Wave Technologies, Inc.). Alternatively, the amount of cinnamolide and/or drimendiol produced may be determined using analytical methods such as gas chromatography/mass spectrometry (GC-MS). Transformed cells and plants can easily be distinguished from non-transformed ones by the presence of the nucleic acid sequence and/or chimeric gene of the invention. The sequences of the plant DNA flanking the insertion site of the transgene can also be sequenced, whereby an “event specific” detection method can be developed, for routine use. See, for example, WO0141558, which describes elite event detection kits (such as PCR detection kits) based for example on the integrated sequence and the flanking (genomic) sequence.

The drimenol oxidase nucleic acid sequence of the invention may be inserted in a plant cell genome so that the inserted coding sequence is downstream (i.e. 3′) of, and under the control of, a promoter which can direct the expression in the plant cell. This is preferably accomplished by inserting the chimeric gene in the plant cell genome, particularly in the nuclear or plastid (e. g. chloroplast) genome.

As the constitutive production of the drimenol oxidase protein may lead to the induction of cell death and/or may lower yield (see e.g. Rizhsky and Mittler, Plant Mol Biol, 2001 46: 313-23), it is in one embodiment preferred to use a promoter whose activity is inducible. Examples of inducible promoters are wound-inducible promoters, such as the MPI promoter described by Cordera et al. (1994, The Plant Journal 6, 141), which is induced by wounding (such as caused by insect or physical wounding), or the COMPTII promoter (WO0056897) or the PR1 promoter described in U.S. Pat. No. 6,031,151. Alternatively the promoter may be inducible by a chemical, such as dexamethasone as described by Aoyama and Chua (1997, Plant Journal 11: 605-612) and in U.S. Pat. No. 6,063,985 or by tetracycline (TOPFREE or TOP 10 promoter, see Gatz, 1997, Annu Rev Plant Physiol Plant Mol Biol. 48: 89-108 and Love et al. 2000, Plant J. 21: 579-88). Other inducible promoters are for example inducible by a change in temperature, such as the heat shock promoter described in U.S. Pat. No. 5,447,858, by anaerobic conditions (e.g. the maize ADH1S promoter), by light (U.S. Pat. No. 6,455,760), by pathogens (e.g. the gst1 promoter of EP759085 or the vst1 promoter of EP309862) or by senescence (SAG12 and SAG13, see U.S. Pat. No. 5,689,042). Obviously, there are a range of other promoters available.

In one embodiment, preferably, an insect pest inducible promoter is used, as thereby the drimenol oxidase protein (or variant or fragment) will only be produced following insect pest attack of the plant tissue. Especially, promoters of genes which are upregulated quickly after insect pest attack are desired. Promoters inducible by a particular plant insect pest may also be identified using known methods, such as cDNA-AFLP®.

Preferably, the promoter is inducible by a number of insect pests, i.e. it is inducible by a broad range of insect pests of the host plant. For each particular host plant species, a different promoter may be most suitable. For example, when tomato is used as a host, the promoter is preferably induced upon at least one, but preferably more than one tomato insect pest. Especially, a promoter which is inducible by one or more insect pests is preferred.

Detailed descriptions of plant insect pests, the disease symptoms caused by them and their life cycles can be found for each plant species. For example, tomato insect pests are described in “Compendium of Tomato Diseases”, Editors Jones, Jones, Stall and Zitter, ISBN 0-89054-120-5, APS Press (http:/www.shopapspress/org).

Alternatively, a host plant may comprise various drimenol oxidase transgenes, each under control of a different pest inducible promoter, to ensure that drimenol oxidase protein is produced following attack by a variety of insect pests. For example, for transformation of tomato, one promoter may be inducible by whitefly and one promoter may be inducible by aphids.

The word “inducible” does not necessarily require that the promoter is completely inactive in the absence of the inducer stimulus. A low level non-specific activity may be present, as long as this does not result in severe yield or quality penalty of the plants. Inducible, thus, preferably refers to an increase in activity of the promoter, resulting in an increase in transcription of the downstream zingiberene synthase coding region following contact with the inducer.

In another embodiment constitutive promoters may be used, such as the strong constitutive 35S promoters or enhanced 35S promoters (the “35S promoters”) of the cauliflower mosaic virus (CaMV) of isolates CM 1841 (Gardner et al., 1981, Nucleic Acids Research 9, 2871-2887), CabbB-S (Franck et al., 1980, Cell 21, 285-294) and CabbB-JI (Hull and Howell, 1987, Virology 86, 482-493); the 35S promoter described by Odell et al. (1985, Nature 313, 810-812) or in U.S. Pat. No. 5,164,316, promoters from the ubiquitin family (e.g. the maize ubiquitin promoter of Christensen et al., 1992, Plant Mol. Biol. 18, 675-689, EP 0 342 926, see also Cornejo et al. 1993, Plant Mol. Biol. 23, 567-581), the gos2 promoter (de Pater et al., 1992 Plant J. 2, 834-844), the emu promoter (Last et al., 1990, Theor. Appl. Genet. 81, 581-588), Arabidopsis actin promoters such as the promoter described by An et al. (1996, Plant J. 10, 107.), rice actin promoters such as the promoter described by Zhang et al. (1991, The Plant Cell 3, 1155-1165) and the promoter described in U.S. Pat. No. 5,641,876 or the rice actin 2 promoter as described in WO070067; promoters of the Cassava vein mosaic virus (WO 97/48819, Verdaguer et al. 1998, Plant Mol. Biol. 37, 1055-1067), the pPLEX series of promoters from Subterranean Clover Stunt Virus (WO 96/06932, particularly the S7 promoter), a alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T-DNA (Velten et al., 1984, EMBO J 3, 2723-2730), the Figwort Mosaic Virus promoter described in U.S. Pat. No. 6,051,753 and in EP426641, histone gene promoters, such as the Ph4a748 promoter from Arabidopsis (PMB 8: 179-191), or others.

Alternatively, a promoter can be utilized which is not constitutive but rather is specific for one or more tissues or organs of the plant (tissue preferred/tissue specific, including developmentally regulated promoters), for example leaf preferred, epidermis preferred, root preferred, flower tissue e.g. tapetum or anther preferred, seed preferred, pod preferred, etc.), or trichome-specific promoters such MTS1 and MSK1 as disclosed in WO2009082208, whereby the drimenol oxidase gene is expressed only in cells of the specific tissue(s) or organ(s) and/or only during a certain developmental stage. For example, the drimenol oxidase gene(s) can be selectively expressed in the leaves of a plant by placing the coding sequence under the control of a light-inducible promoter such as the promoter of the ribulose-1, 5-bisphosphate carboxylase small subunit gene of the plant itself or of another plant, such as pea, as disclosed in U.S. Pat. No. 5,254,799 or Arabidopsis as disclosed in U.S. Pat. No. 5,034,322.

In one embodiment the natural promoter of the drimenol oxidase gene of Persicaria hydropiper provided by the present invention is used. For example, this promoter may be isolated and operably linked to the coding region encoding the drimenol oxidase protein of SEQ ID NO:1. The drimenol oxidase gene promoter (the upstream transcription regulatory region of SEQ ID NO:2) can be isolated from P. hydropiper plants using known methods, such as TAIL-PCR (Liu et al. 1995, Genomics 25(3):674-81; Liu et al. 2005, Methods Mol Biol. 286:341-8), Linker-PCR, or Inverse PCR (IPCR).

The drimenol oxidase coding sequence is preferably inserted into the plant genome so that the coding sequence is upstream (i.e. 5′) of suitable 3′ end nontranslated region (“3′end” or 3′UTR). Suitable 3′ends include those of the CaMV 35S gene (“3′ 35S”), the nopaline synthase gene (“3′ nos”) (Depicker et al., 1982 J. Molec. Appl. Genetics 1, 561-573.), the octopine synthase gene (“3′ocs”) (Gielen et al., 1984, EMBO J 3, 835-845) and the T-DNA gene 7 (“3′ gene 7”) (Velten and Schell, 1985, Nucleic Acids Research 13, 6981-6998), which act as 3′-untranslated DNA sequences in transformed plant cells, and others. In one embodiment the 3′UTR of the P. hydropiper drimenol oxidase gene is used. Introduction of the T-DNA vector into Agrobacterium can be carried out using known methods, such as electroporation or triparental mating.

A drimenol oxidase encoding nucleic acid sequence can optionally be inserted in the plant genome as a hybrid gene sequence whereby the drimenol oxidase sequence is linked in-frame to a (U.S. Pat. No. 5,254,799; Vaeck et al., 1987, Nature 328, 33-37) gene encoding a selectable or scorable marker, such as for example the neo (or nptII) gene (EP 0 242 236) encoding kanamycin resistance, so that the plant expresses a fusion protein which is easily detectable. Alternatively, a drimenol oxidase encoding nucleic acid sequence can be introduced by means of co-tranformation with a gene encoding a selectable or scorable marker, or the two genes can be present on a single T-DNA

All or part of a drimenol oxidase nucleic acid sequence, encoding a drimenol oxidase protein (or variant or fragment), can also be used to transform microorganisms, such as bacteria (e.g. Escherichia coli, Pseudomonas, Agrobacterium, Bacillus, etc.), yeasts, fungi, or algae or insects, or to make recombinant viruses. Transformation of bacteria, with all or part of the drimenol oxidase encoding nucleic acid sequence of this invention, incorporated in a suitable cloning vehicle, can be carried out in a conventional manner, preferably using conventional electroporation techniques as described in Maillon et al. (1989, FEMS Microbiol. Letters 60, 205-210) and WO 90/06999. For expression in prokaryotic host cell, the codon usage of the nucleic acid sequence may be optimized accordingly (as described for plants above). Intron sequences should be removed and other adaptations for optimal expression may be made as known.

The DNA sequence of the drimenol oxidase encoding nucleic acid sequence can be further changed in a translationally neutral manner, to modify possibly inhibiting DNA sequences present in the gene part and/or by introducing changes to the codon usage, e. g., adapting the codon usage to that most preferred by plants, preferably the specific relevant plant genus, as described above.

In one embodiment, several drimenol oxidase encoding nucleic acid sequences are co-expressed in a single host, preferably under control of different promoters. Alternatively, several drimenol oxidase protein encoding nucleic acid sequences can be present on a single transformation vector or be co-transformed at the same time using separate vectors and selecting transformants comprising both chimeric genes. Similarly, one or more drimenol oxidase encoding genes may be expressed in a single plant together with other chimeric genes, for example encoding other proteins which enhance insect pest resistance, or others.

It is understood that the different proteins can be expressed in the same plant, or each can be expressed in a single plant and then combined in the same plant by crossing the single plants with one another. For example, in hybrid seed production, each parent plant can express a single protein. Upon crossing the parent plants to produce hybrids, both proteins are combined in the hybrid plant.

Preferably, for selection purposes but also for weed control options, the transgenic plants of the invention are also transformed with a DNA encoding a protein conferring resistance to herbicide, such as a broad-spectrum herbicide, for example herbicides based on glufosinate ammonium as active ingredient (e.g. Liberty® or BASTA; resistance is conferred by the PAT or bar gene; see EP 0 242 236 and EP 0 242 246) or glyphosate (e.g. RoundUp®; resistance is conferred by EPSPS genes, see e.g. EP 0 508 909 and EP 0 507 698). Using herbicide resistance genes (or other genes conferring a desired phenotype) as selectable marker further has the advantage that the introduction of antibiotic resistance genes can be avoided. Alternatively, other selectable marker genes may be used, such as antibiotic resistance genes. As it is generally not accepted to retain antibiotic resistance genes in the transformed host plants, these genes can be removed again following selection of the transformants. Different technologies exist for removal of transgenes. One method to achieve removal is by flanking the chimeric gene with lox sites and, following selection, crossing the transformed plant with a CRE recombinase-expressing plant (see e.g. EP506763B1). Site specific recombination results in excision of the marker gene. Another site specific recombination systems is the FLP/FRT system described in EP686191 and U.S. Pat. No. 5,527,695. Site specific recombination systems such as CRE/LOX and FLP/FRT may also be used for gene stacking purposes. Further, one-component excision systems have been described, see e.g. WO9737012 or WO9500555).

In a preferred embodiment, the host cell or modified organism of the present invention is capable of producing drimenol and/or drimenol synthase. Suitably, such host cell or modified organism may further comprise a nucleotide sequence encoding a drimenol synthase polypeptide comprising the amino acid sequence of SEQ ID NO:3 or SEQ ID NO:4, or a variant thereof having at least 70%, such as at least 75%, 80%, 85%, 90%, 92%, 95%, 96%, 97%, 98%, 99% or more, sequence identity to the amino acid sequence of SEQ ID NO:3 or SEQ ID NO:4, said drimenol synthase polypeptide having drimenol synthase activity, or a chimeric gene or expression vector comprising such nucleotide sequence encoding a drimenol synthase polypeptide. Variants of the drimenol synthase polypeptide of SEQ ID NO:3 or SEQ ID NO:4 include polypeptides having drimenol synthase activity, which have been derived, by way of one or more amino acid substitutions, deletions or insertions, from the polypeptide having the amino acid sequence of SEQ ID NO:3 or SEQ ID NO:4. Preferably, such variant drimenol synthase polypeptides comprise from 1, 2, 3, 4, 5, 6, 7, 8, 9, 10 or more up to about 100, 90, 80, 70, 60, 50, 45, 40, 35, 30, 25, 20, 15 amino acid substitutions, deletions or insertions as compared to the amino acid sequences of SEQ ID NO:3 or SEQ ID NO:4. In the context of the present invention, the terms “drimenol synthase polypeptide”, “drimenol synthase” and “drimenol synthase polypeptide” include variant and fragments of SEQ ID NO:3 and 4 as set forth above.

In an embodiment, said nucleotide sequence encoding a drimenol synthase polypeptide of the invention is selected from the group consisting of: i) the nucleotide sequence of SEQ ID NO:5 or SEQ ID NO:6; ii) a variant of the nucleotide sequence or a variant of the nucleotide sequence having at least 70%, such as at least 75%, 80%, 85%, 90%, 92%, 95%, 96%, 97%, 98%, 99%, sequence identity to the nucleotide sequence of SEQ ID NO:5 or SEQ ID NO:6 and encoding a polypeptide having drimenol synthase activity; and iii) nucleic acid sequences hybridizing to the complement of SEQ ID NO:5 or SEQ ID NO:6 under stringent hybridization conditions and encoding a polypeptide having drimenol synthase activity.

### Methods and Uses of the Present Invention

In a further aspect, the present invention pertains to the use of an isolated drimenol oxidase polypeptide of the invention, an isolated nucleic acid sequence encoding a drimenol oxidase protein of the present, a chimeric gene comprising such isolated nucleic acid sequence, an expression vector comprising such isolated nucleic acid sequence or chimeric gene, a host cell comprising such isolated nucleic acid sequence, chimeric gene, or expression vector, or a transgenic organism comprising such isolated nucleic acid sequence, chimeric gene, or expression vector, for producing cinnamolide and/or drimendiol. The cinnamolide and/or drimendiol (Zapata et al. 2009. Industrial Crops and Products, vol. 30:119-125) has insecticidal and/or (insect) antifeedant properties.

In another aspect, the present invention relates to a method for producing cinnamolide and/or drimendiol, comprising the steps of: a) contacting drimenol with a drimenol oxidase polypeptide of as taught herein, and b) isolating cinnamolide and/or drimendiol.

In an embodiment, step a) is performed under conditions allowing conversion of drimenol to cinnamolide and/or drimendiol.

Also, the present invention provides a method for preparing cinnamolide and/or drimendiol comprising the steps of: a) transforming or transfecting a host cell with a nucleic acid sequence, chimeric gene or vector of the present invention; b) culturing said host cell under conditions permitting production of cinnamolide and/or drimendiol; and c) optionally, isolating the cinnamolide and/or drimendiol produced in step b). The skilled person will be capable of routinely selecting conditions permitting production of cinnamolide and/or drimendiol. The host cell may have been metabolically engineered (e.g., co-transformed or co-transfected) to further produce or overproduce drimenol, the substrate for the drimenol oxidase of the present invention, to produce cinnamolide and/or drimendiol. The skilled person is capable of accomplishing (over) production of the drimenol based on the drimenol synthase encoding sequences taught herein to (over) produce cinnamolide and/or drimendiol. Similarly, a person skilled in the art will be capable of isolating the cinnamolide and/or drimendiol produced using routine methods for isolation of volatiles. The amino acid sequences of a drimenol synthase polypeptide and variants thereof and nucleotide sequences encoding the same, which may be employed in engineering a host cell to produce or overproduce drimenol, are set forth above.

Similarly, the present invention provides a method for preparing cinnamolide and/or drimendiol comprising the steps of: a) transforming or transfecting a non-human organism, preferably a plant, with a nucleic acid sequence, chimeric gene or vector of the present invention; b) culturing said non-human organism, preferably plant, under conditions permitting production of cinnamolide and/or drimendiol; and c) optionally, isolating the cinnamolide and/or drimendiol produced in step b). The skilled person will be capable of routinely selecting conditions permitting production of cinnamolide and/or drimendiol. The non-human organism may have been metabolically engineered (e.g., co-transformed or co-transfected) to further produce or overproduce drimenol, the substrate for the drimenol oxidase of the present invention, to produce cinnamolide and/or drimendiol. The skilled person is capable of accomplishing overproduction of the drimenol based on the drimenol synthase encoding sequences taught herein to (over) produce cinnamolide and/or drimendiol. Similarly, a person skilled in the art will be capable of isolating the cinnamolide and/or drimendiol produced using routine methods for isolation of volatiles. The amino acid sequences of a drimenol synthase polypeptide and variants thereof and nucleotide sequences encoding the same, which may be employed in engineering a host cell to produce or overproduce drimenol, are set forth above.

The present invention also encompasses a method for producing a drimenol oxidase of the present invention comprising the step of culturing a host cell or growing a transgenic organism comprising at least one drimenol oxidase encoding nucleic acid sequence, chimeric gene or expression vector of the present invention under conditions allowing the production of said drimenol oxidase.

In an aspect, the present invention relates to a method for producing a transgenic plant capable of producing cinnamolide and/or drimendiol, comprising the steps of: a) transforming or transfecting a plant or a plant cell with a drimenol oxidase encoding nucleic acid sequence, chimeric gene comprising the same, or an expression vector comprising the same as taught herein; and b) regenerating a transgenic plant from the transformed or transfected plant or plant cell.

In an alternative embodiment, the method is for producing a transgenic plant capable of producing an enhanced level of cinnamolide and/or drimendiol as compared to a non-transgenic plant of the same genetic background. In an alternative embodiment, the method is for producing a transgenic plant having enhanced insect resistance as compared to a non-transgenic plant of the same genetic background.

In an embodiment, the plant or plant cell is further transformed or transfected or co-transformed or co-transfected with a drimenol synthase encoding nucleic acid sequence as taught herein, or a chimeric gene, or expression vector comprising the same.

In one embodiment, the drimenol oxidase polypeptide of the invention is capable of producing cinnamolide. In another embodiment, the drimenol oxidase polypeptide of the invention is capable of producing drimendiol. In yet another embodiment, the drimenol oxidase of the invention is capable of producing both cinnamolide and drimendiol. In another embodiment, the drimenol oxidase of the present invention is capable of producing several non-identified compounds that are potentially intermediate compounds in the biosynthesis of cinnamolide or conjugated compounds from cinnamolide or other related drimane type sesquiterpenes. Such compounds include, for example, those having a mass fragmentation spectrum as determined by GC-MS essentially as shown in panels A-E in FIG. 1B and those having a mass fragmentation spectrum as determined by GC-MS essentially as shown in panels F-L in FIG. 4B.

Insects Controlled by overexpressing Drimenol oxidase in Plants

An embodiment of the invention provides a method for increasing resistance to a pest insect in a plant by (over) expressing a nucleotide sequence encoding drimenol oxidase or variants thereof as taught herein. Alternatively the invention provides a method for increasing resistance to a pest insect in a plant by further (over) expressing or co-(over) expressing a nucleotide sequence encoding a drimenol synthase or variants thereof as taught herein.

“Insect pest resistance” is an enhanced ability of modified plants of the present invention to withstand attacks of one or more pests compared to wild type or control plants. Methods to assess insect pest resistance in plants are known in the art. For example, disease symptoms may be scored visually at one or more time points after infestation or contact with an insect pest. Alternatively, insect pests may be detected and optionally quantified during infestation of the plants in an assay. A modified plant shows enhanced pest resistance if the number of insect pests detected on the plant, e.g., on a specific tissue of the plant, is significantly lower compared to the number of insects detected on a control (non-modified) plant of the same genetic background. Preferably, a significant increase in average yield of modified plants of the present invention compared to control (e.g. at least 1%, 2%, 5%, 10% or more) when plants are grown under insect pest pressure provides an indirect measurement of enhanced resistance to pest insects. Statistical analyses may be employed to determine existence of significant differences.

The invention now having been fully described is exemplified in the Examples below and in the claims, which are not to be construed as further limiting. References cited herein are hereby incorporated by reference in their entirety.

### BRIEF DESCRIPTION OF THE DRAWINGS

FIG. 1A shows the GC-MS chromatograms of dichloromethane extracts of leaves infiltrated with RBCS-PhDS (P. hydropiper drimenol synthase) and c11763-E2 (P. hydropiper drimenol oxidase) together with AtFPS2 and AtHMGR precursor genes, and a control empty vector pBinPlus construct. FIG. 1B shows the mass fragmentation spectra of all the novel peaks (see panels A-E) identified in FIG. 1A. However, the nature of all these novel peaks could not be identified with a NIST library search. The mass fragmentation spectra of polygodial is shown as a reference.

FIG. 2 shows the results of the analysis by Liquid Chromatography-Quadruple Time-of-Flight Mass Spectrometry/Mass Spectrometer (LC-QTOF-MS/MS) of methanolic extracts of N. benthamiana leaves infiltrated with RBCS-PhDS (P. hydropiper drimenol synthase), c11763-E2 (P. hydropiper drimenol oxidase) and both genes co-infiltrated simultaneously, together with the AtFPS2 and AtHMGR precursor genes. Y-axis scale is identical in all chromatograms. All the major novel compounds detected in leaves co-infiltrated with both genes have been indicated by arrows. The novel compound eluting at Rt 41.50 min (MH+235.15) which has a similar mass as the major novel compound detected by GCMS (m/z 234) has been purified and has been identified as cinnamolide by NMR analysis.

FIG. 3 shows the proposed pathway from FPP and drimenol to cinnamolide and polygodial including formulas and calculated masses. Cinnamolide (in plant extract) and drimendiol (in yeast extract) was identified by NMR spectroscopy.

FIG. 4A shows the results for the Gas Chromatography-Mass Spectra Analysis (GCMS) of the ethyl acetate extract of the medium in which the yeast strain WAT11 expressing PhDS (P. hydropiper drimenol synthase) and c11763-E2 (P. hydropiper drimenol oxidase) was grown. Many novel compounds were produced, some of which similar as detected in the N. benthamiana (FIG. 1), but also some yeast specific compounds. One of the major peaks in the gas chromatogram could be attributed to drimendiol. FIG. 4B shows the mass fragmentation spectra of all the novel peaks (see panels F-L) identified in FIG. 4A. However, the nature of these novel peaks could not be identified with a NIST library search.

FIG. 5 shows the results of the analysis by Liquid Chromatography-Quadruple Time-of-Flight Mass Spectrometry/Mass Spectrometer (LC-QTOF-MS/MS) of yeast extract co-transformed with PhDS (drimenol synthase) and c11763-E2 (drimenol oxidase). The major compound eluting at Rt 36.55 min was purified and identified as drimendiol by NMR analysis.

FIG. 6 shows the effect of increasing concentrations of cinnamolide on the feeding behaviour at 6 h from the start of the experiment in aphids (Myzus persicae) (see top panel) and white flies (Bemisia tabaci (see bottom panel). Antifeedant index (Al %)=[(C−T)/(C+T)]×100. Positive values indicate an antifeedant effect while negative values indicate a phagostimulant effect. Error bars indicate standard error, 6 replicates for aphids and 8 for whiteflies, with an average of 98 whiteflies and 20 aphids per replicate.

Sequence Listing

SEQ ID NO: 1 Drimenol oxidase (protein) from Persicaria hydropiper
DYLILICIVVSGLLLYSSRTSKMKLPPGPPGLPIVGNLFDLGSLPHRSLAKLAKLHGPVCLRMG
RLRVVVISSDSAAKEVLQTSDTLFCNRFVYDSLTASQHHTFSMALLPPTALWKSLRKISASQL
FTNARMNASQHLRRKQLEDLLSYVESCSRSGTAINIAQAAFNTSVNLLSKTFFSVDLIDPSSS
NSVEFKEMVWQIMLESGTPNLADYFPVLRRIDPQGNRRRMKIQFEKILDLFNTMIRQRLDEK
GGCFDEINDTLDALLKINQDNSEELDLSVIPHLLLDLFVGGSESTSSTVEWAMALLFSNPEKM
KKAKEELETVVGKGIAVKEEDTGRLPYLQAAIKETFRMHPPTPFLIPRKTDSDVDLCGFTVQK
GSQVIVNAWAIGRDPSLWENADTFEPERFLGMEIDVKGRNFELIPFGAGRRICPGLPIAMRM
LTLMVANLINCFEWRLEGGAAPETLDMSDKIGFTLQRAHPFRVIPTSIIQGCDVSTALNN
SEQ ID NO: 2 Nucleic acid sequence (cDNA) encoding Drimenol oxidase
from Persicaria hydropiper
ATGGATTACTTGATCTTGATGATTTGCATTGTGGTGTCAGGGTTACTGCTCTACTCATCAA
GAACGAGCAAGATGAAGCTTCCTCCGGGGCCTCCAGGACTGCCCATCGTGGGGAATCT
CTTCGACCTCGGAAGCCTGCCGCACAGGTCCTTAGCCAAGCTCGCCAAGCTCCATGGG
CCTGTCATGTGCCTCCGGATGGGTCGTCTACGGGTGGTCGTTATCTCTTCCGATTCCGC
GGCGAAAGAAGTCCTTCAGACGAGCGATACTTTGTTCTGCAACAGGTTCGTCTACGACT
CCCTCACCGCTCCCAACACCACACTTTCTCGATGGCGTTATTGCCCCCCACCGCTCTGT
GGAAGAGCCTCCGCAAGATCTCCGCCTCCCAGTTGTTCACAAATGCCCGGATGAACGC
CAGCCAGCATCTCCGGCGGAAGCAGCTGGAGGACCTCCTCTCCTATGTAGAGAGCTGC
AGCCGGAGTGGGACTGCCATTAACATCGCTCAAGCTGCATTCAATACCTCCGTGAATCT
CCTGTCAAAAACTTTCTTCTCCGTTGATCTTATAGACCCGAGTTCGAGCAACTCCGTTGA
ATTCAAGGAGATGGTGTGGCAGATAATGTTGGAATCTGGTACACCGAACTTAGCTGACT
ATTTTCCGGTTCTTAGGAGAATTGATCCACAGGGGAATAGGCGTCGGATGAAGATCCAA
TTTGAGAAGATCCTCGACCTTTTCAATACTATGATTCGTCAACGACTTGACGAGAAAGGA
GGTTGCTTTGATGAAATCAATGACACTTTGGATGCTTTGCTCAAGATTAACCAAGATAATA
GTGAGGAGCTTGATCTTTCCGTTATCCCACATCTACTATTGGACTTGTTCGTCGGAGGAA
GCGAATCGACGTCAAGCACGGTTGAATGGGCAATGGCGTTGCTGTTTTCCAACCCGGA
GAAGATGAAGAAAGCTAAAGAAGAGCTCGAAACCGTAGTTGGGAAAGGCATTGCAGTCA
AGGAGGAAGATACCGGCAGGCTACCGTACTTGCAAGCTGCAATAAAAGAGACTTTCAGG
ATGCATCCACCAACCCCATTTTTGATCCCAAGGAAAACGGATTCCGATGTTGATCTTTGT
GGGTTCACAGTGCAAAAGGGCTCGCAAGTGATCGTCAATGCGTGGGCTATAGGGCGAG
ATCCAAGCTTGTGGGAGAATGCTGACACCTTTGAACCGGAGAGGTTTTTGGGTATGGAG
ATTGATGTTAAAGGCCGTAACTTTGAGCTGATCCCGTTCGGTGCTGGACGGAGAATCTG
CCCTGGGCTACCAATAGCAATGAGAATGCTTACTCTGATGGTAGCCAATTTGATTAACTG
CTTTGAATGGAGGCTTGAAGGTGGGGCTGCACCAGAAACCTTAGATATGAGCGATAAAA
TCGGATTCACACTTCAAAGAGCTCATCCCTTCCGAGTTATCCCTACCAGCATCATCCAGG
GATGTGATGTTTCCACAGCTCTTAATAATTGA
SEQ ID NO: 3 Drimenol synthase (protein) from Valeriana officinalis
MSTALNSEHETVRPLASFQPSTWGDLFISYSEDSQLKEVYGNEHECLKQQVKTMLLDVTNY
RISEKIAFINTLERLGVSHEFENEIEGLLHQMFDAHSKFQDGIQHFDLFTLGIYFRILRQHGYRI
YCDVFNKLKDSNNEFKKELKEDAIGLLSLYEATQVRAHAEEILDEALIFTKAQLESIAATSSLSP
FVEKQITHALVQALHKGIPRVESRHFISVYEEDPDKNDLLLRFSKIDYNIVQMLHKQELCHISK
WWRDSELETKLTYARNRVAECFLWTLCVYHEPKYSPARLLLGKLINIISCTDDTYDAYGTLEE
VQIFTDVIQRLDRSSMEQLPDYMKILYKAVLDLFDEVEVQLSNQETNNTYRMAYAKEELKAIA
KCYEKEHIWFRKCHVPPFEEYLENAVVSIGNRLAVTFSFLGMDQVAAVEAFEWAKTDPKMV
KSCGKVLRLVDDVMSHEEEDVRGHVATGVECYMKEHGVSREEAVVEFYKRVEYAWKDVN
EEFITPNHLHDLLNRVLNLTRIADVVYKFEDGYTHPEKTLKHHIMALFVDPVPV
SEQ ID NO: 4 Drimenol synthase (protein) from Persicaria hydropiper
MSTAVNVPSAVRPADKRPIASFHPSPWGDYFLKYVPCDQVTQAKMEDEVKKVEEDVKKELR
KLAKAVGKPLELLNFIDVVERLGVGYRLEQEIEDLVQAIFDNDKFGVDEFDLYHTSLWFRLLR
QHGFHVSCDVFGKFKGRNGRFKDSLASDVKGILGLYEASHVRTHGDDTLDEALVFTTTHLK
AVVTNQPNHPLVPQVTHALMQPYHKGMPRLESRHFIAFYEKDPYHDKTLLKFGKLDFNLVQ
ALHKKELKDLSRWWKDLDMHAKMPFPSRDRVPEGFWTLGPFYEPQFALCRKFFLQVFKVT
SIVDDIYDAYGTIDELTAFTKAAERWDRSCLDELPEYMKVSYASLIDTFEEFERDLAPQGRSW
SVKYAREEMIQMCRVYYQEAKWCHEKYSPTCDEYLEKASIVSFGYNLGTVVCFLGMGDVAT
KEAFEWARGNPKVVRAAGIIGRLMDDIGSHHFEQGRDHVPSAVECYIRQHGVDEVTAQREL
GKRVESSWKDINEMMLKPYMMPKPLLTRILNECRIVDVIYKGEDSYTFSNTTMKKNISHILTD
PIPI
SEQ ID NO: 5 Nucleic acid sequence (cDNA) encoding Drimenol synthase
from Valeriana officinalis
ATGTCTACTGCATTAAACAGTGAGCATGAAACTGTTCGTCCATTAGCAAGTTTTCAACCA
AGTACATGGGGCGATCTTTTCATCTCTTATTCTGAAGATAGCCAGCTTAAGGAAGTATAT
GGTAATGAGCACGAATGTCTGAAACAACAAGTGAAAACAATGTTGTTGGATGTGACAAAT
TATAGAATTTCCGAGAAAATCGCTTTCATAAATACGTTGGAGAGATTAGGGGTATCTCAT
GAGTTTGAGAATGAGATTGAAGGTCTGCTTCATCAAATGTTTGATGCTCATTCTAAATTCC
AAGATGGTATTCAACACTTTGATTTGTTCACATTGGGGATTTACTTCAGGATTCTCAGGCA
ACATGGCTATAGAATCTATTGTGATGTTTTCAACAAGTTGAAAGATAGCAACAATGAATTC
AAGAAGGAACTTAAAGAGGACGCGATCGGTTTGCTAAGTTTGTACGAAGCAACACAAGT
AAGAGCACACGCTGAAGAAATTTTAGACGAAGCCCTCATTTTCACAAAGGCTCAACTTGA
ATCCATAGCCGCAACCTCCAGCTTAAGCCCATTTGTCGAGAAGCAAATTACTCATGCTTT
GGTCCAAGCTCTCCACAAAGGAATCCCAAGAGTCGAATCGCGCCATTTCATCTCTGTTTA
TGAAGAAGATCCTGACAAAAATGATTTGTTGTTGAGGTTCTCAAAGATTGATTACAATATT
GTACAAATGCTTCACAAGCAAGAATTGTGCCATATCTCAAAGTGGTGGAGAGATTCGGA
GCTCGAAACAAAACTAACTTATGCGAGGAATAGAGTGGCGGAATGCTTTTTATGGACTCT
TTGTGTGTACCACGAACCAAAGTACTCTCCGGCTCGGCTTCTGTTAGGCAAACTCATAAA
TATCATATCTTGCACTGATGACACATATGATGCGTATGGTACATTAGAGGAAGTTCAGAT
CTTTACAGATGTCATACAAAGGTTGGATAGGAGTTCTATGGAGCAGCTGCCGGATTACAT
GAAAATCCTCTACAAAGCTGTCCTTGATCTTTTCGACGAAGTAGAAGTTCAGCTATCGAA
CCAAGAAACTAATAATACTTATCGTATGGCTTATGCCAAGGAAGAGTTAAAAGCTATCGC
CAAGTGCTACGAAAAGGAGCACATATGGTTCAGAAAATGTCACGTGCCCCCATTCGAAG
AATATCTAGAGAATGCGGTAGTGTCAATCGGTAATCGTTTGGCCGTAACTTTTTCTTTTCT
GGGAATGGATCAAGTAGCAGCTGTTGAAGCGTTCGAGTGGGCCAAAACTGATCCCAAAA
TGGTAAAATCGTGCGGTAAAGTCTTACGACTTGTTGACGACGTAATGAGCCACGAGGAG
GAAGATGTAAGAGGACACGTGGCAACGGGAGTCGAATGCTACATGAAAGAACACGGAG
TGAGTAGGGAAGAGGCCGTCGTGGAGTTCTACAAGAGGGTCGAGTACGCGTGGAAGGA
TGTGAACGAGGAATTTATAACGCCGAACCATCTGCATATCGACCTCCTCAACCGCGTTCT
TAACCTTACAAGAATTGCAGACGTTGTTTACAAGTTTGAAGACGGCTACACGCACCCCGA
GAAGACTCTGAAACATCATATCATGGCGTTGTTCGTCGACCCCGTCCCCGTATAGA
SEQ ID NO: 6 Nucleic acid sequence (cDNA) encoding Drimenol synthase
from Persicaria hydropiper
ATGTCTACTGCCGTTAACGTCCCATCTGCGGTCCGCCCCGCCGACAAGCGTCCGATTGC
GAGCTTTCACCCGAGCCCATGGGGCGACTACTTCCTCAAATACGTTCCTTGTGACCAGG
TGACTCAAGCCAAGATGGAAGATGAGGTGAAGAAAGTTGAAGAGGATGTAAAGAAGGAG
TTGCGGAAGCTGGCGAAGGCTGTAGGGAAGCCATTGGAGCTGCTCAACTTCATCGATGT
CGTCGAACGCCTTGGGGTGGGATACCGCCTTGAGCAGGAGATCGAGGACCTTGTTCAA
GCTATATTCGACAACGACAAATTTGGAGTCGATGAATTCGATCTCTATCATACTTCCCTCT
GGTTTCGCCTCCTTAGGCAACATGGGTTTCACGTATCATGTGATGTGTTCGGAAAATTCA
AGGGCAGAAACGGAAGGTTCAAGGACTCGTTGGCGAGTGATGTGAAGGGGATACTCGG
CTTGTACGAAGCCTCACATGTTCGCACCCATGGCGATGACACGCTTGATGAAGCATTGG
TGTTTACTACGACTCATCTTAAAGCCGTAGTGACCAACCAACCAAACCATCCCTTGGTGC
CACAAGTGACCCATGCCCTAATGCAGCCCTACCACAAGGGCATGCCAAGGCTCGAGTCT
AGGCACTTCATCGCATTCTACGAGAAAGATCCTTACCACGACAAAACCTTGTTGAAATTT
GGCAAATTGGACTTCAACTTGGTGCAAGCATTGCACAAGAAGGAGCTCAAAGATCTCAG
CAGGTGGTGGAAAGATCTAGATATGCACGCGAAGATGCCTTTCCCGAGCAGAGACCGA
GTGCCCGAAGGCTACTTTTGGACACTAGGGCCTTTCTATGAACCACAATTCGCTCTTTGT
CGAAAATTTTTCTTGCAAGTGTTCAAAGTAACTTCCATTGTCGATGATATCTACGATGCCT
ATGGAACTATCGATGAGCTCACCGCTTTCACTAAAGCTGCTGAGAGATGGGATCGTAGT
TGCCTTGATGAACTTCCGGAATACATGAAAGTGAGCTACGCGTCTCTCATTGATACCTTC
GAGGAATTTGAACGCGACTTGGCTCCCCAAGGAAGATCTTGGAGCGTCAAGTACGCAAG
AGAGGAAATGATACAGATGTGTAGAGTTTACTACCAAGAAGCGAAATGGTGCCATGAGA
AATACTCGCCCACCTGCGACGAGTACTTGGAGAAAGCATCCATAGTGAGTTTCGGCTAC
AACTTGGGAACAGTAGTGTGCTTCCTCGGGATGGGAGACGTCGCTACAAAGGAGGCATT
CGAATGGGCTCGCGGAAACCCAAAGGTCGTAAGAGCCGCAGGCATAATCGGAAGGCTC
ATGGACGACATAGGCAGCCATCATTTTGAGCAAGGTAGAGACCATGTTCCATCCGCCGT
GGAGTGCTACATAAGGCAGCACGGTGTCGACGAAGTAACCGCCCAAAGAGAGTTGGGA
AAGCGGGTGGAAAGTAGCTGGAAGGACATCAATGAGATGATGTTGAAGCCTTATATGAT
GCCGAAGCCTCTTCTAACTCGCATCCTTAACGAGTGTCGCATTGTGGATGTGATCTACAA
GGGAGAAGATAGCTACACCTTCTCCAAC

### EXAMPLES

### Example 1

### Cloning of Drimenol Oxidase cDNA from P. hydropiper

The full length cDNA of the drimenol oxidase gene (SEQ ID NO:2) was cloned into the yeast expression vector pYEDP60 (Pompon D, Louerat B, Bronine A, Urban P (1996). Method Enzymol. Vol. 272:51-64), which was modified to contain PacI and NotI sites at the polylinker using the NotI/PacI restriction sites. The drimenol oxidase cDNA was co-transformed with drimenol synthase cDNA from P. hydropiper into yeast strain WAT 11. After transformation, yeast clones expressing the PhDS alone or PhDS combined with the drimenol oxidase were selected on Synthetic Dextrose minimal medium (0.67% Difco yeast nitrogen base medium without amino acids, 2% D-glucose, 2% agar) supplemented with amino acids, but omitting L-tryptophane or uracil, adenine sulphate and L-tryptophane, respectively, for auxotrophic selection of transformants.

### Example 2

### Co-Expression of Drimenol Synthase cDNA (P. hydropiper) with Drimenol Oxidase cDNA (P. hydropiper) in Yeast

A starter yeast culture was grown overnight at 30° C. in 5 ml of Synthetic Galactose minimal medium (0.67% Difco yeast nitrogen base medium without amino acids, 2% d-galactose, amino acids, but omitting L-tryptophane or uracil, adenine sulphate and L-tryptophane). The starter culture was diluted to OD600 of 0.05 in 50 ml of Synthetic Galactose minimal medium and incubated at 200 rpm at 30° C. The culture was grown for 3 days and extracted with 10 ml ethyl acetate From this, a sample was analyzed by GC-MS.

### Example 3

### Plasmid Construction for Expression of Cinnamolide and/or Drimendiol in Nicotiana benthamiana

For transient expression in N. benthamiana, drimenol synthase cDNA and drimenol oxidase cDNA were cloned into ImpactVector1.1 (http://www.impactvector.com/) to express them under the control of the Rubisco (RBC) promoter. The drimenol synthase cDNA was also cloned into ImpactVector 1.4 and 1.5 to fuse it with the RBC promoter and the plastid or CoxIV mitochondrial targeting sequence. An LR reaction (Gateway-LR Clonase TM II) was carried out to clone each gene into pBinPlus binary vector between the right and left borders of the T-DNA for plant transformation. The vectors were introduced in Agrobacterium tumefaciens.

### Example 4

### Transient Expression of Cinnamolide and/or Drimendiol in Leaves of Nicotiana benthamiana

The drimenol synthase cDNA and drimenol oxidase cDNA were co-infiltrated with farnesyl diphosphate synthase (AtFPS) and 3-hydroxy-3-methylglutaryl-CoA reductase (AtHMGR), which are known to greatly increase the expression levels of some terpenoids (Van Herpen et al, PLoS One. 2010; 5(12):e14222). To this end, the Agrobacterium strains were grown at 28° C. at 220 rpm for 24 hours in LB media with kanamycin (50 mg/L) and rifampicillin (100 mg/L). Cells were harvested by centrifugation for 20 min at 4000 g and 20° C. and then resuspended in 10 mM MES buffer containing 10 mM MgCl2 and 100 μM acetosyringone (4′-hydroxy-3′,5′-dimethoxyacetophenone, Sigma) to a final OD600 of 0.5, followed by incubation at room temperature and 50 rpm for 3 hours.

For co-infiltration, equal volumes of the Agrobacterium strains were mixed. Nicotiana benthamiana plants were grown from seeds on soil in a greenhouse with 16 h light at 28° C. (16 h)/25° C. (8 h). Strain mixtures were infiltrated into leaves of four-week-old N. benthamiana plants using a 1 mL syringe. The bacteria were slowly injected into the abaxial side of the leaf. The plants were grown and infiltrated leaves were collected 5 days after infiltration. Compounds accumulated in the leaves were analyzed by snap freezing and grinding 500 mg infiltrated leaf from plant in liquid nitrogen and extraction with 2 ml dichloromethane. The extracts were prepared by brief vortexing and sonication for 5 min. Then the extracts were centrifuged for 10 min at 1200 rpm and the clear part of the solution was transferred to a fresh vial. Finally, the extracts were concentrated by evaporating the solvent to a volume of about 0.5 mL and dehydrated using anhydrous Na2SO4. Analysis of the samples was performed by GC-MS (Agilent GC-MS, Agilent technologies).

### Example 5

### Gas Chromatography-Mass Spectra Analysis (GC-MS)

Analytes from 1 μL samples were separated using a gas chromatograph (5890 series II, Hewlett-Packard) equipped with a 30 m×0.25 mm, 0.25 mm film thickness column (ZB-5, Phenomenex) using helium as carrier gas at flow rate of 1 ml/min. The injector was used in splitless mode with the inlet temperature set to 250° C. The initial oven temperature of 45° C. was increased after 1 min to 300° C. at a rate of 10° C./min and held for 5 min at 300° C. The GC was coupled to a mass-selective detector (model 5972A, Hewlett-Packard). Compounds were identified by comparison of mass spectra and retention times (rt) with those of the authentic standards.

### Example 6

### Analysis by Liquid Chromatography-Quadruple Time-of-Flight Mass Spectometry/Mass Spectrometer (LC-QTOF-MS/MS)

Liquid chromatography, coupled to quadruple time-of-flight mass spectrometry (LC-QTOF-MS) was performed using a Waters Alliance 2795 HPLC connected to a Waters 2996 PDA detector and subsequently a QTOF Ultima V4.00.00 mass spectrometer (Waters, MS technologies, UK) operating in positive ionization mode. The column used was an analytical column (Luna 3μ C18/2 100 A; 2.0×150 mm; Phenomenex, USA) attached to a C18 pre-column (2.0×4 mm; AJO-4286; Phenomenex, USA). Degassed eluent A (ultra-pure water:formic acid (10001, v/v)) and eluent B (acetonitril:formic acid (10001, v/v)) were used at 0.19 ml·min-1. The gradient started at 5% B and increased linearly to 65% B in 45 min, after which the column was washed and equilibrated for 15 min before the next injection. The injection volume was 5 μl.

### Example 7

### NMR Spectroscopy

NMR analysis was performed at Spinnovations BV (Nijmegen) on a Bruker Avance III 500 MHz spectrometer equipped with a 5-mm CPTCI cryo probe (1H-13C/15N/2H+Z-gradients) operating at 303 K. The structure identification with polygodial as a reference compound was based on a 1D 1H, 1H-1H-DQF-COSY, 1H-1H-TOCSY, 1H-1H-NOESY, 1H-13C-HSQC and 1H-13CHMBC spectra. The structure identification of the purified compound was based on the same selection of NMR experiments with the exception of the 1H-13C-HMBC spectrum due to the limited amount of material. The proton and carbon chemical shifts were referenced to the internal reference TMS (proton, d=0.00 ppm; carbon, d=0.00 ppm). The data were processed using Topspin 2.1 pl5.

### Example 8

### Cinnamolide Purification

Cinnamolide was purified from a plant methanolic extract, obtained from transiently transformed N. benthamiana plants expressing the drimenol synthase cDNA and the drimenol oxidase cDNA. The extract was evaporated and injected onto a C18 analytical column. The HPLC was performed using a water:acetonitrile gradient starting at 40% ACN and ending at 75% ACN. After identification of the desired fraction by UV absorbance, it was collected multiple times until a sufficient amount was obtained. The resulting water:ACN mixture was evaporated using a SpeedVac. The collected fractions were re-dissolved in methanol and pooled together stepwise as their volume decreased in subsequent evaporation steps; in the last step the crystalline compound was weighed and finally dissolved in 96% ethanol.

### Example 9

### Insect Preparation

The insects used in this study were the silverleaf whitefly Bemisia tabaci (genotype B) and the green peach aphid Myzus persicae. Whiteflies were reared on tomato (Moneymaker cultivar) in a greenhouse at 26° C., at 60% relative humidity with a photoperiod of 16 h light and 8 h dark. To perform the assays, adult flies of both genders were collected from leaves with an aspirator. They were cold-anesthetized at 7° C. in a 3 cm diameter plastic cylinder covered with Parafilm® for 5 min, before being released into the Petri dish at the start of the dual-choice assay, to ensure they would not fly out before the dishes were sealed. Apterous (without wings) aphid adults were collected from Chinese cabbage (Brassica rapa L. subspecies pekinensis), on which they had been reared at room temperature in ambient light. They were collected in 2 ml test tubes and starved for about 30 min before inoculating them in the dishes.

### Example 10

### Insect Choice Assays

In the dual-choice assays, insects were presented with circular tomato leaf areas coated with either 50% ethanol in water (control) or a solution of cinnamolide in 50% ethanol in water (treatment; two disks with each solution). Cinnamolide was firstly dissolved in 96% ethanol at a concentration of 2 mg mL⁻¹; subsequently an equal volume of water was added bringing the maximum concentration used to 1 mg mL⁻¹. Further dilutions were performed adding directly one volume of 50% ethanol in water to one volume of the previous concentration. The three concentrations used were therefore 1 mg mL⁻¹, 500 μg mL⁻¹ and 250 μg mL⁻¹. Fresh Moneymaker tomato leaves collected from 5 or 6 weeks old plants were cut in half longitudinally and placed abaxial-side up in a 9-cm-Petri dish on 45 ml of 8 g L⁻¹ water agar substrate. The leaves were then covered with a Petri dish bottom through which four holes of 16 mm diameter were drilled, making sure that each half leaf had two circular areas available for insects to feed upon. On each circular area (disk), 15 μl of solution were applied and spread using a size 3 watercolor brush. The control and treatment solutions were each spread on two diametrically opposed disks to control for potential environmental cues, such as light.

The insects were introduced into the dishes after the solvent had evaporated. Insects were put onto the lid of the dishes and then covered with the bottom of the Petri dish containing the leaves. In that way all insects had to fly or walk to the feeding areas without being mistakenly put closer to one disk than to another. On average, 98 whiteflies and 20 aphids per plate were used for each assay. For each combination of insect species and compound concentration, eight biological replicates eight separate arenas) were used. The plates were sealed with Parafilm® and kept upside down. The assays with aphids were carried out at room temperature on a lab bench (uncontrolled conditions). The whiteflies assays were performed in a climate chamber at 25° C., 60% humidity and 16/8 h light/dark photoperiod.

The assays were carried on for either 18 or 24 h and the feeding insects counted at 30 min, 1 h, 2 h, 6 h, and 18 h for whiteflies and at 30 min, 1 h, 2 h, 6 h, and 24 h for aphids. Insects were considered to be feeding when they were immobile on one spot on a leaf disk. Five leaf disks were weighed to be able to determine the concentration of the insect repellent compounds in relationship to fresh weight (FW) of the leaf area treated. The average mass of each disk was found to be 34 mg. To convert data from the concentration in ethanol, expressed as mg mL⁻¹, into concentration expressed as mg gFW⁻¹, the value must be multiplied by 15 (the volume in μL applied on each disk) and divided by 0.034 (mass of leaf disk in grams). To convert the data into μg cm⁻², the original concentration must be again multiplied by 15 and then divided by 1.6 (area of the disk in cm²). We estimated that the concentrations of coated cinnamolide correspond to approximately 450, 225 and 113 μg gFW⁻¹.

### Example 11

### Data Analysis

To express the repellence potency of cinnamolide towards the two insect species tested, the Antifeedant Index (Al %) was calculated according to Kutas and Nádasy, as follows: Al %=[(C−T)/(C+T)]×100, where C indicates the number of insects feeding on the control, and T the number of insects feeding on the treatment (Kutas and Nádasy, 2005). The Al % assumes positive values when the tested compound is an antifeedant, and negative values when the compound is a phagostimulant (i.e. stimulate feeding). To determine the ED₅₀, the effective dose at which 50% of the insects are deterred (i.e. the concentration at which twice as many insects feed on the control compared to the treatment), a Probit analysis was carried out using the PASW statistics 18 (SPSS Inc.) package.

### Results

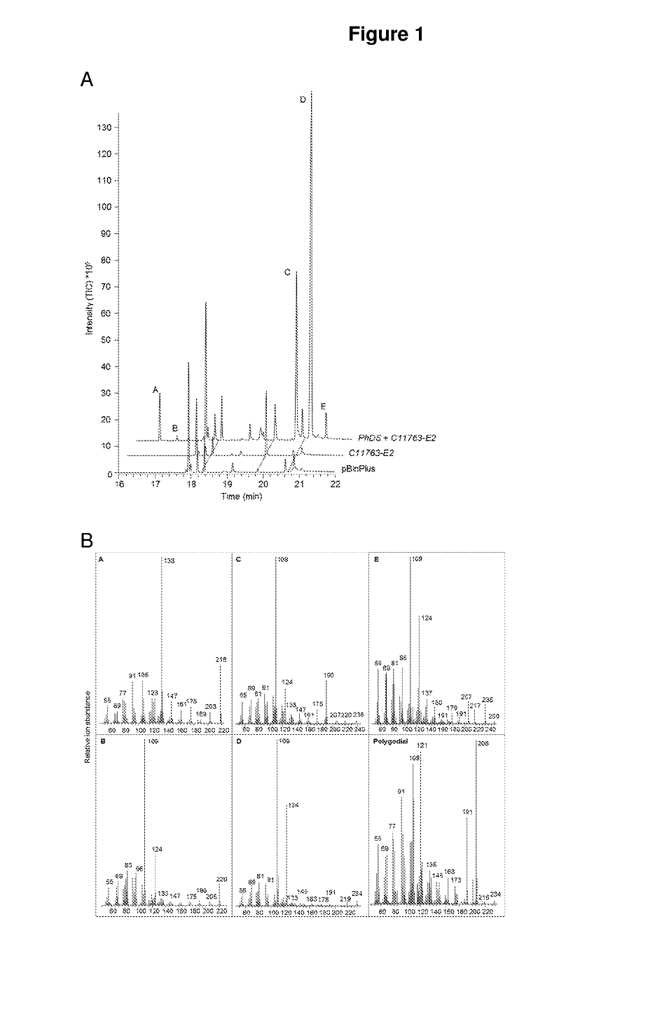

FIG. 1. GC-MS chromatogram of dichloromethane extracts of N. benthamiana leaves infiltrated with RBCS-PhDS (drimenol synthase) and c11763-E2 P450 (P. hydropiper drimenol oxidase) and including control experiments. In N. benthamiana, co-expression of the PhDS (drimenol synthase) together with the c11763-E2 P450 (drimenol oxidase) revealed several novel compounds in leaf extracts analyzed by GCMS compared with leaves infiltrated with the c11763-E2 (drimenol oxidase) or the empty vector control (pBinPlus). None of the novel compounds showed high homology to the polygodial mass spectra (used as a reference compound) and therefore could not be identified with a NIST library search. However, the novel compounds showed similarity to drimenol. Some of the masses of the novel compounds corresponded to the calculated masses of intermediates in the polygodial or cinnamolide pathway.

Analysis by Liquid Chromatography-Quadruple Time-of-Flight Mass Spectometry/Mass Spectrometer (LC-QTOF-MS/MS) of N. benthamiana Extract

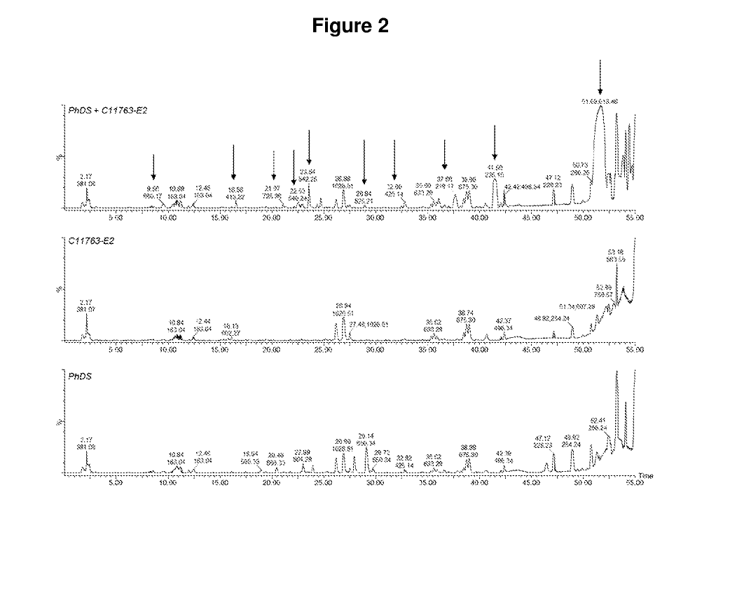

FIG. 2 displays LC-MS chromatograms of methanol extracts of N. benthamiana leaves agro-infiltrated with AtHMGR, AtFPS2, PhDS (drimenol synthase) and c11763-E2 P450 (drimenol oxidase) including control experiment. The novel compound eluting at Rt 41.5 min, which has a similar mass as the major novel compound detected by GCMS (m/z 234), was purified and could be identified as cinnamolide by NMR analysis. Several other novel compounds were detected by LC-MS which have not been identified by NMR. In literature, it is described that polygodial very quickly forms adducts with —NH2 or —SH groups (Cimino et al., 1984; Ischia et al., 1982), e.g., it can form a conjugate with any peptide or protein. We cannot exclude that besides cinnamolide, polygodial may also be produced. Some of the unknown novel compounds detected by LC-MS could be conjugated cinnamolide, polygodial or other related drimane type sesquiterpenes.

### Proposed Pathway to Cinnamolide and Polygodial

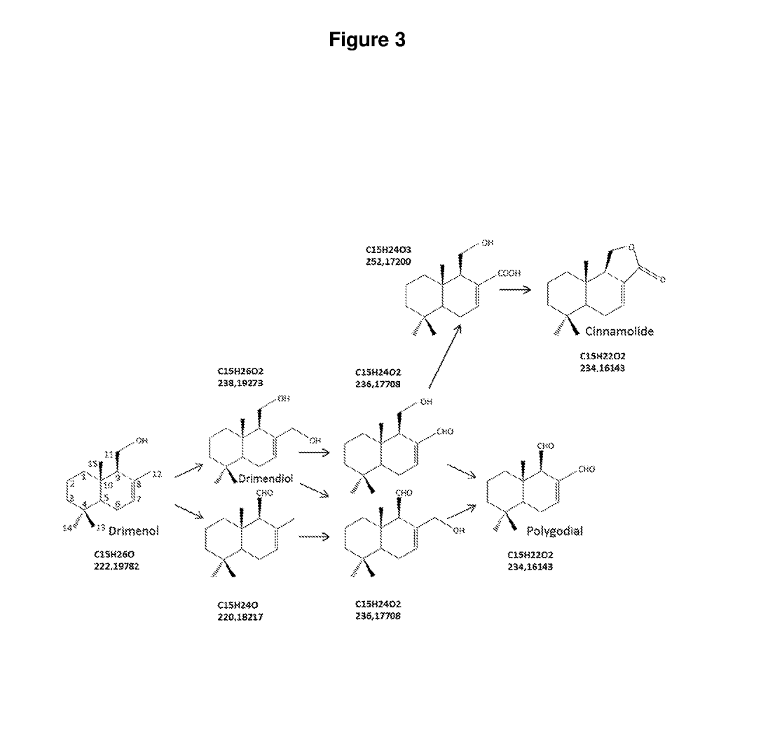

FIG. 3 displays the proposed biosynthesis pathway of cinnamolide and polygodial. Cinnamolide represents one of the major novel compound (234 m/z) in methanol extract from N. benthamiana leaves that were agro-infiltrated with AtHMGR, AtFPS2, PhDS (drimenol synthase) and c11763-E2 P450 (drimenol oxidase). The structure of cinnamolide was identified by NMR (Spinnovation BV). Drimendiol represents a major novel compound detected in a medium extract of yeast co-transformed with the drimenol synthase and the drimenol oxidase. The structure of drimendiol has also been identified by NMR.

### Gas Chromatography-Mass Spectra Analysis (GCMS) Yeast Medium Extract

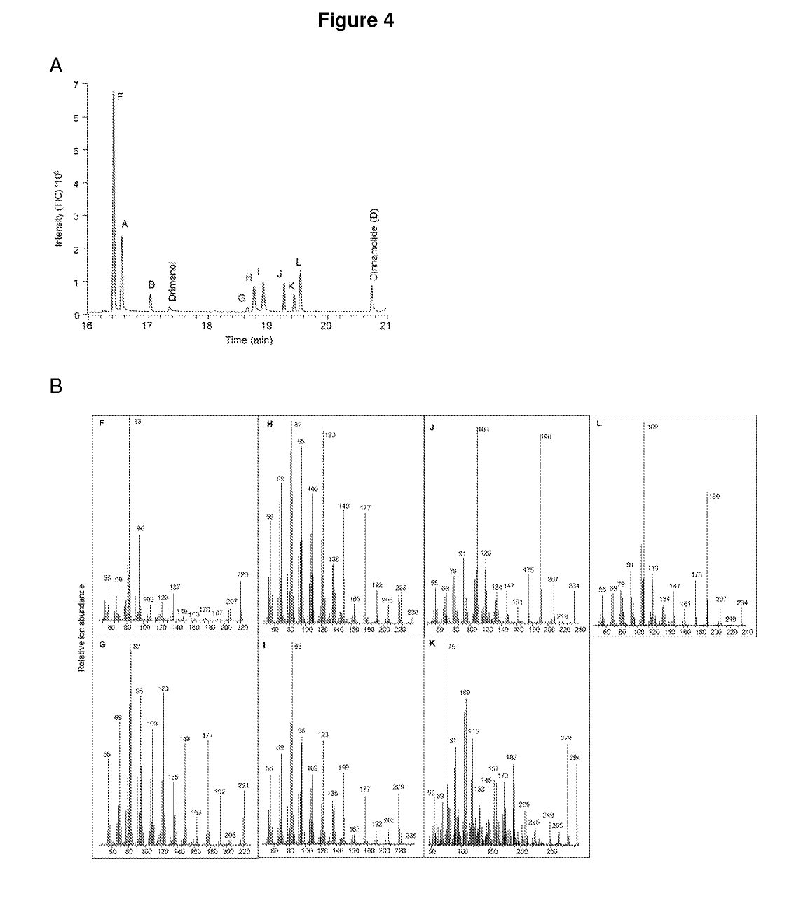

FIG. 4 displays GCMS Chromatogram of ethyl acetate extract from the yeast strain WAT11 expressing PhDS (drimenol synthase) and c11763-E2 P450 (drimenol oxidase) from P. hydropiper (see FIG. 4A). The results show that many novel compounds are produced, some of which similar as detected in N. benthamiana (FIG. 1, compound A and B) but also some yeast specific compounds (see FIG. 4B, panels F-L). The results also show that in yeast cinnamolide is not the major novel compound produced. All novel compounds shown in FIG. 4B could not be identified with a NIST library search.

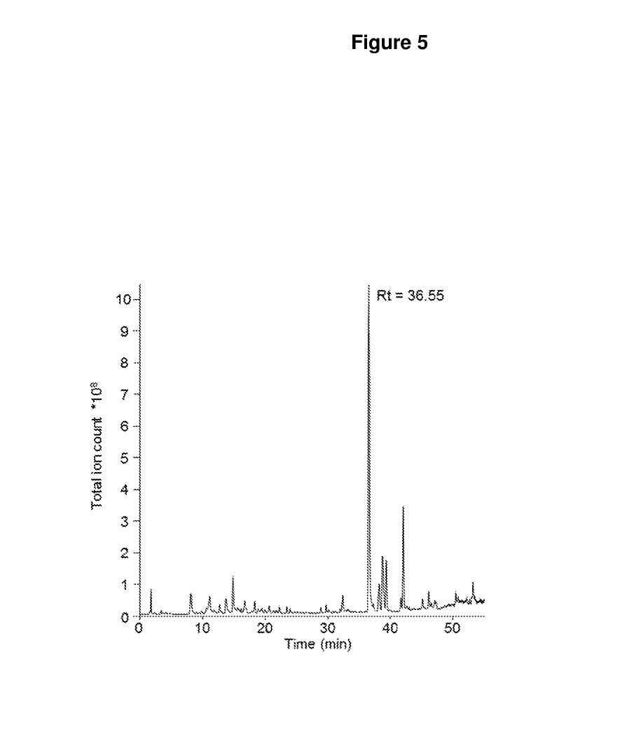

FIG. 5 shows the results of the analysis by Liquid Chromatography-Quadruple Time-of-Flight Mass Spectrometry/Mass Spectrometer (LC-QTOF-MS/MS) of yeast extract co-transformed with PhDS (drimenol synthase) and c11763-E2(drimenol oxidase). The major compound eluting at Rt 36.55 min has been purified and identified as drimendiol by NMR analysis (Bisqualis, Wageningen UR).

Bioassays with Insects

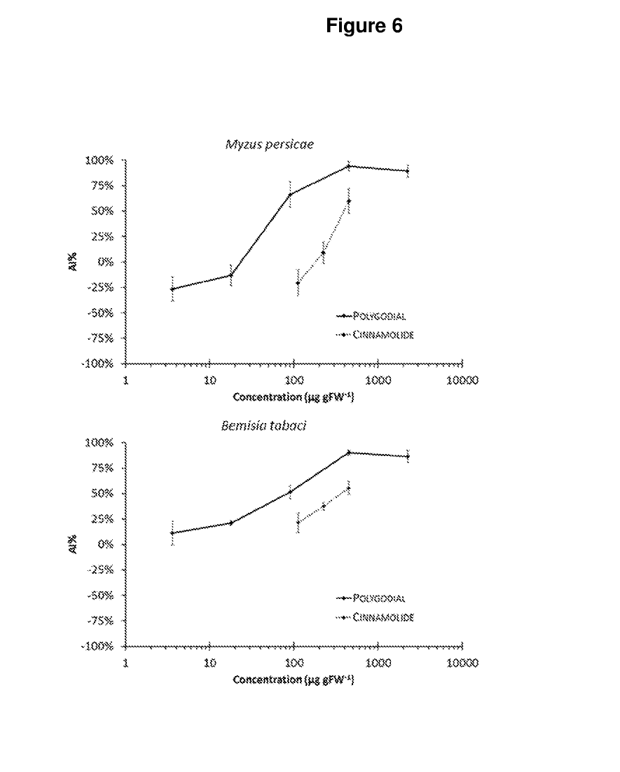

FIG. 6 displays the effects of cinnamolide on aphids (top panel) and whiteflies (bottom panel) tested in dual-choice assays. The preference for the treated or control areas was assessed using three different concentrations of cinnamolide, namely 450, 225 and 113 μg gFW⁻¹. Dose-dependent responses in feeding preference were recorded for a period of 18 or 24 hours post inoculation with aphids or whiteflies, respectively. The 6 h-time point was chosen as the most reliable figure as most insects were actively feeding at that time, and the time for induction of potentially confounding secondary responses was kept at a minimum. The feeding preference was assessed using the Antifeedant Index (Al %). The results reveal that the Al % of cinnamolide increases with the increasing concentrations applied, as seen in FIG. 5, meaning that there is a clear dose-dependent preference for the control disks over the cinnamolide-treated ones. Similarly to what was shown for polygodial (used as a reference compound), cinnamolide displayed an attractant (phagostimulant) effect on aphids at the lowest concentration used. This effect was not seen in the case of whiteflies. The extrapolated effective deterrent dose for 50% feeding deterrence (ED₅₀) was 195 μg gFW⁻¹ for whiteflies and 423 μg gFW⁻¹ for aphids.

1.-35. (canceled)

36. An isolated polypeptide having drimenol oxidase activity and comprising an amino acid sequence selected from the group consisting of SEQ ID NO:1, and an amino acid sequence having at least 70% identity with the amino acid sequence of SEQ ID NO:1.

37. An isolated nucleic acid sequence encoding the polypeptide according to claim 36.

38. The isolated nucleic acid sequence according to claim 37 comprising a nucleotide sequence of SEQ ID NO: 2, or a nucleotide sequence having at least 70% sequence identity with the nucleotide sequence of SEQ: ID NO:2.

39. The isolated nucleic acid sequence according to claim 37 derived from Persicaria hydropiper.

40. A chimeric gene comprising the nucleic acid sequence according claim 37.

41. An expression vector comprising the nucleic acid sequence according to claim 37.

42. The expression vector according to claim 41, operably linked to at least one regulatory sequence which controls transcription, translation initiation or termination.

43. The expression vector according to claim 41, wherein the nucleic acid sequence further comprises a targeting sequence.

44. The expression vector according to claim 43, wherein the targeting sequence encodes a transit peptide that is capable of targeting the polypeptide having drimenol oxidase activity to a plastid of the plant cell.

45. The expression vector according to claim 44, wherein the plastid is a chloroplast.

46. The expression vector according to claim 43, wherein the targeting sequence encodes a transit peptide that targets the polypeptide having drimenol oxidase activity to a mitochondrion of the plant cell.

47. A host cell comprising the isolated nucleic acid sequence according to claim 37.

48. The host cell according to claim 47, wherein the cell is a mammalian cell, a bacterial cell, a fungal cell, or a plant cell.

49. A host cell according to claim 47, further comprising a nucleotide sequence encoding a polypeptide comprising the amino acid sequence of SEQ ID NO:3 or SEQ ID NO:4, or a variant thereof having at least 70% sequence identity to the amino acid sequence of SEQ ID NO:3 or SEQ ID NO:4, or a chimeric gene or vector comprising a nucleotide sequence encoding a polypeptide comprising the amino acid sequence of SEQ ID NO: 3 or SEQ ID NO:4, or a variant of thereof having at least 70% sequence identity with the amino acid sequence of SEQ ID NO:3 or SEQ ID NO:4, said polypeptide having drimenol synthase activity.

50. A host cell according to claim 47, further comprising the nucleotide sequence of SEQ: ID NO:5, or SEQ ID NO:6 or a variant thereof having at least 70% sequence identity to the nucleotide sequence of SEQ ID NO:5 or SEQ ID NO:6, or a chimeric gene comprising the nucleotide sequence of SEQ ID NO: 5 or SEQ ID NO:6, or a variant of thereof having at least 70% sequence identity with the nucleotide sequence of SEQ ID NO:5 or SEQ ID NO:6.

51. A transgenic organism comprising the isolated nucleic acid sequence according to claim 37.

52. The transgenic organism according to claim 51, wherein the organism is a plant.

53. A transgenic organism according to claim 51, further comprising a nucleotide sequence encoding a polypeptide comprising the amino acid sequence of SEQ ID NO:3 or SEQ ID NO:4, or a variant thereof having at least 70% sequence identity to the amino acid sequence of SEQ ID NO:3 or SEQ ID NO:4.

54. A method for producing cinnamolide and/or drimendiol, comprising:
(a) contacting drimenol with a polypeptide according to claim 36 under conditions allowing conversion of drimenol to cinnamolide and/or drimendiol;
(b) isolating said cinnamolide and/or drimendiol.

55. The method according to claim 54, further comprising, prior to step (a): transfecting and/or transforming a host cell capable of producing drimenol and/or drimenol synthase with a nucleic acid according to claim 2, to provide for a host cell capable of producing cinnamolide and/or drimendiol.

56. The method according to claim 54, wherein the host cell is further transfected and/or transformed with a nucleic acid sequence encoding a polypeptide comprising the amino acid sequence of SEQ ID NO:3 or SEQ ID NO:4, or a variant thereof having at least 70% sequence identity to the amino acid sequence of SEQ ID NO:3 or SEQ ID NO:4, or a chimeric gene comprising a nucleotide sequence encoding a polypeptide comprising the amino acid sequence of SEQ ID NO: 3 or SEQ ID NO:4, or a variant of thereof having at least 70% sequence identity with the amino acid sequence of SEQ ID NO:3 or SEQ ID NO:4, said polypeptide having drimenol synthase activity.

57. The method according to claim 54, wherein the host cell is further transfected and/or transformed with the nucleotide sequence of SEQ: ID NO:5 or SEQ ID NO:6, or variant thereof having at least 70% sequence identity to the nucleotide sequence of SEQ ID NO:5 or SEQ ID NO:6, or a chimeric gene comprising the nucleotide sequence of SEQ ID NO: 5 or SEQ ID NO:6, or a variant of thereof having at least 70% sequence identity with the nucleotide sequence of SEQ ID NO:5 or SEQ ID NO:6.

58. The method according to claim 54, wherein step (a) is carried out by culturing the host cell under conditions allowing production of cinnamolide and/or drimendiol.

59. The method according to claim 54, wherein the host cell is a plant cell, bacterial cell or fungal cell.

60. A method for producing a polypeptide having drimenol oxidase activity, comprising:
(a) transforming or transfecting a host cell or a non-human organism with a nucleic acid sequence according to claim 37;
(b) culturing the host cell or the organism under conditions allowing production of the polypeptide.

61. The method according to claim 60, wherein the host cell or non-human organism further comprises a nucleotide sequence encoding a polypeptide comprising the amino acid sequence of SEQ ID NO:3 or SEQ ID NO:4, or a variant thereof having at least 70% sequence identity to the amino acid sequence of SEQ ID NO:3 or SEQ ID NO:4, or a chimeric gene comprising a nucleotide sequence encoding a polypeptide comprising the amino acid sequence of SEQ ID NO: 3 or SEQ ID NO:4, or a variant of thereof having at least 70% sequence identity with the amino acid sequence of SEQ ID NO:3 or SEQ ID NO:4, said polypeptide having drimenol synthase activity.

62. The method according to claim 60, wherein the host cell or the non-human cell further comprises the nucleotide sequence of SEQ: ID NO:5 or SEQ ID NO:6, or variant thereof having at least 70% sequence identity to the nucleotide sequence of SEQ ID NO:5 or SEQ ID No:6, or a chimeric gene comprising the nucleotide sequence of SEQ ID NO: 5 or SEQ ID NO:6, or a variant of thereof having at least 70% sequence identity with the nucleotide sequence of SEQ ID NO:5 or SEQ ID NO:6.

63. A method for producing a transgenic plant capable of producing cinnamolide and/or drimendiol, comprising:
(a) transforming or transfecting a plant or a plant cell with a nucleic acid sequence according to claim 37;
(b) regenerating a transgenic plant from the transformed or transfected plant or plant cell.

64. A method according to claim 63, wherein the plant or plant cell further comprises a nucleotide sequence encoding a polypeptide comprising the amino acid sequence of SEQ ID NO:3 or SEQ ID NO:4, or a variant thereof having at least 70% sequence identity to the amino acid sequence of SEQ ID NO:3 or SEQ ID NO:4, or a chimeric gene comprising a nucleotide sequence encoding a polypeptide comprising the amino acid sequence of SEQ ID NO: 3 or SEQ ID NO:4, or a variant of thereof having at least 70% sequence identity with the amino acid sequence of SEQ ID NO:3 or SEQ ID NO:4, said polypeptide having drimenol synthase activity.

65. A method according to claim 63, wherein the plant or plant cell further comprises the nucleotide sequence of SEQ: ID NO:5 or SEQ ID NO:6, or variant thereof having at least 70% sequence identity to the nucleotide sequence of SEQ ID NO:5 or SEQ ID NO:6, or a chimeric gene comprising the nucleotide sequence of SEQ ID NO:5 or SEQ ID NO:6, or a variant of thereof having at least 70% sequence identity with the nucleotide sequence of SEQ ID NO:5 or SEQ ID NO:6.

66. The method according to claim 63, further comprising:
(c) screening the transgenic plant, or a plant derived therefrom by selfing or crossing, for production of cinnamolide and/or drimendiol and identifying a transgenic plant producing cinnamolide and/or drimendiol.

67. A method of conferring pest insect resistance to a crop plant, comprising expressing cinnamolide in the crop plant.

68. The method of claim 67, wherein the pest insects are sap suckling insects.

69. A crop plant comprising an isolated nucleic acid sequence according to claim 37.

70. A crop plant according to claim 68, which is selected from tomato, potato, peppers, ornamental species, maize/corn, wheat, barley, oat, sorghum, rye, soybean, cotton, Brassica spp., rice, and vegetable species.


SEQUENCE LISTING (6 sequences)

SEQ ID NO: 1 - protein, 503 aa, Persicaria hydropiper
MDYLILMICIVVSGLLLYSSRTSKMKLPPGPPGLPIVGNLFDLGSLPHRSLAKLAKLHGPVMCLRMGRLRVVVISSDSAAKEVLQTSDTLFCNRFVYDSLTASQHHTFSMALLPPTALWKSLRKISASQLFTNARMNASQHLRRKQLEDLLSYVESCSRSGTAINIAQAAFNTSVNLLSKTFFSVDLIDPSSSNSVEFKEMVWQIMLESGTPNLADYFPVLRRIDPQGNRRRMKIQFEKILDLFNTMIRQRLDEKGGCFDEINDTLDALLKINQDNSEELDLSVIPHLLLDLFVGGSESTSSTVEWAMALLFSNPEKMKKAKEELETVVGKGIAVKEEDTGRLPYLQAAIKETFRMHPPTPFLIPRKTDSDVDLCGFTVQKGSQVIVNAWAIGRDPSLWENADTFEPERFLGMEIDVKGRNFELIPFGAGRRICPGLPIAMRMLTLMVANLINCFEWRLEGGAAPETLDMSDKIGFTLQRAHPFRVIPTSIIQGCDVSTALNN

SEQ ID NO: 2 - DNA, 1512 bp, Persicaria hydropiper
atggattacttgatcttgatgatttgcattgtggtgtcagggttactgctctactcatcaagaacgagcaagatgaagcttcctccggggcctccaggactgcccatcgtggggaatctcttcgacctcggaagcctgccgcacaggtccttagccaagctcgccaagctccatgggcctgtcatgtgcctccggatgggtcgtctacgggtggtcgttatctcttccgattccgcggcgaaagaagtccttcagacgagcgatactttgttctgcaacaggttcgtctacgactccctcaccgcctcccaacaccacactttctcgatggcgttattgccccccaccgctctgtggaagagcctccgcaagatctccgcctcccagttgttcacaaatgcccggatgaacgccagccagcatctccggcggaagcagctggaggacctcctctcctatgtagagagctgcagccggagtgggactgccattaacatcgctcaagctgcattcaatacctccgtgaatctcctgtcaaaaactttcttctccgttgatcttatagacccgagttcgagcaactccgttgaattcaaggagatggtgtggcagataatgttggaatctggtacaccgaacttagctgactattttccggttcttaggagaattgatccacaggggaataggcgtcggatgaagatccaatttgagaagatcctcgaccttttcaatactatgattcgtcaacgacttgacgagaaaggaggttgctttgatgaaatcaatgacactttggatgctttgctcaagattaaccaagataatagtgaggagcttgatctttccgttatcccacatctactattggacttgttcgtcggaggaagcgaatcgacgtcaagcacggttgaatgggcaatggcgttgctgttttccaacccggagaagatgaagaaagctaaagaagagctcgaaaccgtagttgggaaaggcattgcagtcaaggaggaagataccggcaggctaccgtacttgcaagctgcaataaaagagactttcaggatgcatccaccaaccccatttttgatcccaaggaaaacggattccgatgttgatctttgtgggttcacagtgcaaaagggctcgcaagtgatcgtcaatgcgtgggctatagggcgagatccaagcttgtgggagaatgctgacacctttgaaccggagaggtttttgggtatggagattgatgttaaaggccgtaactttgagctgatcccgttcggtgctggacggagaatctgccctgggctaccaatagcaatgagaatgcttactctgatggtagccaatttgattaactgctttgaatggaggcttgaaggtggggctgcaccagaaaccttagatatgagcgataaaatcggattcacacttcaaagagctcatcccttccgagttatccctaccagcatcatccagggatgtgatgtttccacagctcttaataattga

SEQ ID NO: 3 - protein, 555 aa, Valeriana officinalis
MSTALNSEHETVRPLASFQPSTWGDLFISYSEDSQLKEVYGNEHECLKQQVKTMLLDVTNYRISEKIAFINTLERLGVSHEFENEIEGLLHQMFDAHSKFQDGIQHFDLFTLGIYFRILRQHGYRIYCDVFNKLKDSNNEFKKELKEDAIGLLSLYEATQVRAHAEEILDEALIFTKAQLESIAATSSLSPFVEKQITHALVQALHKGIPRVESRHFISVYEEDPDKNDLLLRFSKIDYNIVQMLHKQELCHISKWWRDSELETKLTYARNRVAECFLWTLCVYHEPKYSPARLLLGKLINIISCTDDTYDAYGTLEEVQIFTDVIQRLDRSSMEQLPDYMKILYKAVLDLFDEVEVQLSNQETNNTYRMAYAKEELKAIAKCYEKEHIWFRKCHVPPFEEYLENAVVSIGNRLAVTFSFLGMDQVAAVEAFEWAKTDPKMVKSCGKVLRLVDDVMSHEEEDVRGHVATGVECYMKEHGVSREEAVVEFYKRVEYAWKDVNEEFITPNHLHDLLNRVLNLTRIADVVYKFEDGYTHPEKTLKHHIMALFVDPVPV

SEQ ID NO: 4 - protein, 558 aa, Persicaria hydropiper
MSTAVNVPSAVRPADKRPIASFHPSPWGDYFLKYVPCDQVTQAKMEDEVKKVEEDVKKELRKLAKAVGKPLELLNFIDVVERLGVGYRLEQEIEDLVQAIFDNDKFGVDEFDLYHTSLWFRLLRQHGFHVSCDVFGKFKGRNGRFKDSLASDVKGILGLYEASHVRTHGDDTLDEALVFTTTHLKAVVTNQPNHPLVPQVTHALMQPYHKGMPRLESRHFIAFYEKDPYHDKTLLKFGKLDFNLVQALHKKELKDLSRWWKDLDMHAKMPFPSRDRVPEGFWTLGPFYEPQFALCRKFFLQVFKVTSIVDDIYDAYGTIDELTAFTKAAERWDRSCLDELPEYMKVSYASLIDTFEEFERDLAPQGRSWSVKYAREEMIQMCRVYYQEAKWCHEKYSPTCDEYLEKASIVSFGYNLGTVVCFLGMGDVATKEAFEWARGNPKVVRAAGIIGRLMDDIGSHHFEQGRDHVPSAVECYIRQHGVDEVTAQRELGKRVESSWKDINEMMLKPYMMPKPLLTRILNECRIVDVIYKGEDSYTFSNTTMKKNISHILTDPIPI

SEQ ID NO: 5 - DNA, 1672 bp, Valeriana officinalis
atgtctactgcattaaacagtgagcatgaaactgttcgtccattagcaagttttcaaccaagtacatggggcgatcttttcatctcttattctgaagatagccagcttaaggaagtatatggtaatgagcacgaatgtctgaaacaacaagtgaaaacaatgttgttggatgtgacaaattatagaatttccgagaaaatcgctttcataaatacgttggagagattaggggtatctcatgagtttgagaatgagattgaaggtctgcttcatcaaatgtttgatgctcattctaaattccaagatggtattcaacactttgatttgttcacattggggatttacttcaggattctcaggcaacatggctatagaatctattgtgatgttttcaacaagttgaaagatagcaacaatgaattcaagaaggaacttaaagaggacgcgatcggtttgctaagtttgtacgaagcaacacaagtaagagcacacgctgaagaaattttagacgaagccctcattttcacaaaggctcaacttgaatccatagccgcaacctccagcttaagcccatttgtcgagaagcaaattactcatgctttggtccaagctctccacaaaggaatcccaagagtcgaatcgcgccatttcatctctgtttatgaagaagatcctgacaaaaatgatttgttgttgaggttctcaaagattgattacaatattgtacaaatgcttcacaagcaagaattgtgccatatctcaaagtggtggagagattcggagctcgaaacaaaactaacttatgcgaggaatagagtggcggaatgctttttatggactctttgtgtgtaccacgaaccaaagtactctccggctcggcttctgttaggcaaactcataaatatcatatcttgcactgatgacacatatgatgcgtatggtacattagaggaagttcagatctttacagatgtcatacaaaggttggataggagttctatggagcagctgccggattacatgaaaatcctctacaaagctgtccttgatcttttcgacgaagtagaagttcagctatcgaaccaagaaactaataatacttatcgtatggcttatgccaaggaagagttaaaagctatcgccaagtgctacgaaaaggagcacatatggttcagaaaatgtcacgtgcccccattcgaagaatatctagagaatgcggtagtgtcaatcggtaatcgtttggccgtaactttttcttttctgggaatggatcaagtagcagctgttgaagcgttcgagtgggccaaaactgatcccaaaatggtaaaatcgtgcggtaaagtcttacgacttgttgacgacgtaatgagccacgaggaggaagatgtaagaggacacgtggcaacgggagtcgaatgctacatgaaagaacacggagtgagtagggaagaggccgtcgtggagttctacaagagggtcgagtacgcgtggaaggatgtgaacgaggaatttataacgccgaaccatctgcatatcgacctcctcaaccgcgttcttaaccttacaagaattgcagacgttgtttacaagtttgaagacggctacacgcaccccgagaagactctgaaacatcatatcatggcgttgttcgtcgaccccgtccccgtataga

SEQ ID NO: 6 - DNA, 1680 bp, Persicaria hydropiper
atgtctactgccgttaacgtcccatctgcggtccgccccgccgacaagcgtccgattgcgagctttcacccgagcccatggggcgactacttcctcaaatacgttccttgtgaccaggtgactcaagccaagatggaagatgaggtgaagaaagttgaagaggatgtaaagaaggagttgcggaagctggcgaaggctgtagggaagccattggagctgctcaacttcatcgatgtcgtcgaacgccttggggtgggataccgccttgagcaggagatcgaggaccttgttcaagctatattcgacaacgacaaatttggagtcgatgaattcgatctctatcatacttccctctggtttcgcctccttaggcaacatgggtttcacgtatcatgtgatgtgttcggaaaattcaagggcagaaacggaaggttcaaggactcgttggcgagtgatgtgaaggggatactcggcttgtacgaagcctcacatgttcgcacccatggcgatgacacgcttgatgaagcattggtgtttactacgactcatcttaaagccgtagtgaccaaccaaccaaaccatcccttggtgccacaagtgacccatgccctaatgcagccctaccacaagggcatgccaaggctcgagtctaggcacttcatcgcattctacgagaaagatccttaccacgacaaaaccttgttgaaatttggcaaattggacttcaacttggtgcaagcattgcacaagaaggagctcaaagatctcagcaggtggtggaaagatctagatatgcacgcgaagatgcctttcccgagcagagaccgagtgcccgaaggctacttttggacactagggcctttctatgaaccacaattcgctctttgtcgaaaatttttcttgcaagtgttcaaagtaacttccattgtcgatgatatctacgatgcctatggaactatcgatgagctcaccgctttcactaaagctgctgagagatgggatcgtagttgccttgatgaacttccggaatacatgaaagtgagctacgcgtctctcattgataccttcgaggaatttgaacgcgacttggctccccaaggaagatcttggagcgtcaagtacgcaagagaggaaatgatacagatgtgtagagtttactaccaagaagcgaaatggtgccatgagaaatactcgcccacctgcgacgagtacttggagaaagcatccatagtgagtttcggctacaacttgggaacagtagtgtgcttcctcgggatgggagacgtcgctacaaaggaggcattcgaatgggctcgcggaaacccaaaggtcgtaagagccgcaggcataatcggaaggctcatggacgacataggcagccatcattttgagcaaggtagagaccatgttccatccgccgtggagtgctacataaggcagcacggtgtcgacgaagtaaccgcccaaagagagttgggaaagcgggtggaaagtagctggaaggacatcaatgagatgatgttgaagccttatatgatgccgaagcctcttctaactcgcatccttaacgagtgtcgcattgtggatgtgatctacaagggagaagatagctacaccttctccaacaccaccatgaagaaaaacatttctcacattctcaccgatcccattccgatttag